(19) United States
(12) Patent Application Publication
(10) Pub. No.: US 2026/0158003 A1
(43) Pub. Date: Jun. 11, 2026

(54) METHODS TO TARGET THE SOLUBLE UROKINASE PLASMINOGEN ACTIVATOR RECEPTOR PATHWAY FOR THE PREVENTION AND TREATMENT OF ATHEROSCLEROSIS

(71) Applicant: The Regents of the University of Michigan, Ann Arbor, MI (US)

(72) Inventor: Salim HAYEK, Ann Arbor, MI (US)

(21) Appl. No.: 18/707,511

(22) PCT Filed: Nov. 4, 2022
(86) PCT No.: PCT/US2022/079295

§ 371 Date: May 3, 2024

Related U.S. Application Data
(60) Provisional application No. 63/275,586, filed on Nov. 4, 2021.
(60) Provisional application No. 63/377,653, filed on Sep. 29, 2022.

(51) Int. Cl. A61K 31/4164; G01N 33/68

(52) U.S. Cl. CPC A61K 31/4164; G01N 33/6893; G01N 2800/323

(57) ABSTRACT

Provided herein are methods for preventing or treating atherosclerosis by reducing levels or inhibiting the activity of soluble urokinase plasminogen activator receptor (suPAR) protein and/or and downstream effectors thereof (e.g., v 3 integrin, vitronectin, uPA, etc.) in a subject. The method comprising determining the level of soluble urokinase plasminogen activator receptor (suPAR) protein in a sample obtained from a subject; and treating the subject to reduce suPAR levels and/or inhibit suPAR activity if the suPAR level in the sample is elevated, wherein treating the subject to reduce suPAR protein levels and/or inhibit the activity of suPAR comprises administering an anti-suPAR therapy in the form of a nucleic acid inhibitor, an antisense oligonucleotide (ASO), an siRNA, an shRNA, an element of a as/CRISPR system or an antibody or antibody fragment that binds to suP AR or a ligand or receptor of suP AR and thereby inhibits the activity of suPAR.

### CROSS-REFERENCE TO RELATED APPLICATION

The present application claims priority to U.S. Provisional Patent Application Ser. No. 63/275,586, filed Nov. 4, 2021, and U.S. Provisional Patent Application Ser. No. 63/377,653, filed Sep. 29, 2022, both of which are hereby incorporated by reference in their entireties.

### SEQUENCE LISTING

The text of the computer readable sequence listing filed herewith, titled “UM_40075_253_SequenceListing.xml,” created May 9, 2024, having a file size of 6,775 bytes, is hereby incorporated by reference in its entirety.

### FIELD

Provided herein are methods for preventing or treating atherosclerosis by reducing levels or inhibiting the activity of soluble urokinase plasminogen activator receptor (suPAR) protein and/or and downstream effectors thereof (e.g., αvβ3 integrin, vitronectin, uPA, etc.) in a subject.

### BACKGROUND

Despite major progress in the control of cardiovascular risk factors such as hypertension, high cholesterol and diabetes mellitus, heart disease remains the number one cause of death in the United States and worldwide. Systemic inflammation is recognized as a key process driving cardiovascular disease and is estimated to account for at least 30% of the residual risk in patients on maximal treatment. There are no currently approved therapies targeting inflammation in patients with cardiovascular disease.

### SUMMARY

Provided herein are methods for preventing or treating atherosclerosis by reducing levels or inhibiting the activity of soluble urokinase plasminogen activator receptor (suPAR) protein and/or and downstream effectors thereof (e.g., αvβ3 integrin, vitronectin, uPA, etc.) in a subject.

People with kidney disease are disproportionately affected by atherosclerosis for unclear reasons. Soluble urokinase plasminogen activator receptor (suPAR) is an immune-derived mediator of kidney disease which levels are strongly associated with cardiovascular outcomes. Experiments were conducted during development of embodiments herein to assess suPAR's pathogenic involvement in atherosclerosis using epidemiologic, genetic, and experimental approaches. It was found that suPAR levels are predictive of coronary artery calcification and cardiovascular events in 5,406 participants of the Multi-Ethnic Study of Atherosclerosis (MESA). In a genome-wide association meta-analysis including 12,937 participants, a missense variant was identified in the PLAUR gene (rs4760) and confirmed experimentally to lead to higher suPAR levels. Mendelian randomization analysis in the UK Biobank using rs4760 indicated a causal association between genetically predicted suPAR levels and atherosclerotic phenotypes. Lastly, in a mouse model of atherosclerosis using proprotein convertase subtilisin/kexin type 9 serine protease (PCSK9) transfection, mice over-expressing suPAR (suPAR^{Tg}) had dramatically increased atherosclerotic plaques with necrotic cores compared to wild-type mice. Exposure to high suPAR levels induces pro-inflammatory changes in monocyte profile and function, priming the immune system to accelerate atherosclerosis in response to other injuries. These findings characterize suPAR as a shared pathogenic factor between atherosclerosis and kidney disease.

In some embodiments, provided herein are methods of treating or preventing atherosclerosis in a subject, comprising treating the subject to reduce soluble urokinase plasminogen activator receptor (suPAR) protein levels and/or inhibit the activity of suPAR or its downstream effectors. In some embodiments, the subject exhibits elevated levels of suPAR. In some embodiments, the subject exhibits elevated levels of suPAR in the blood or a processed blood product (e.g., plasma, serum, etc.). In some embodiments, the subject suffers from atherosclerosis. In some embodiments, the subject is at elevated risk of atherosclerosis based on one or more risk factors (e.g., age, family history, genetics, biomarkers, sex, lifestyle, etc.). In some embodiments, treating the subject to reduce suPAR protein levels and/or inhibit the activity of suPAR and downstream effectors comprises administering an anti-suPAR therapy to the subject. In some embodiments, the anti-suPAR therapy inhibits the expression of suPAR. In some embodiments, the anti-suPAR therapy is a nucleic acid inhibitor of suPAR expression. In some embodiments, the nucleic acid inhibitor of suPAR expression is an antisense oligonucleotide (ASO), an siRNA, an shRNA, or an element of a Cas/CRISPR system. In some embodiments, the anti-suPAR therapy inhibits the activity of suPAR or its downstream effector. In some embodiments, the anti-suPAR therapy is an antibody or antibody fragment that binds to suPAR or a ligand or receptor of suPAR and thereby inhibits the activity of suPAR. In some embodiments, the anti-suPAR therapy is a peptide or small molecule that binds to suPAR and thereby inhibits the activity of suPAR. In some embodiments, the small molecule is azeliragon, which antagonizes the Receptor for Advanced Glycation Endproducts (RAGE), a cofactor for suPAR.

In some embodiments, provided herein are methods of assessing and treating/preventing atherosclerosis in a subject, comprising: (a) determining the level of soluble urokinase plasminogen activator receptor (suPAR) protein in a sample obtained from a subject; and (b) treating the subject to reduce excess suPAR levels if the suPAR level in the sample is elevated. In some embodiments, the sample is a blood sample, plasma sample, serum sample, etc. In some embodiments, methods further comprise comparing the level of suPAR to a threshold level to determine if the level of suPAR is elevated. In some embodiments, the threshold level is based on an algorithm that incorporates one or more risk factors of atherosclerosis (e.g., age, renal function, gender lifestyle, biomarkers, family history, medical history, etc.). In some embodiments, excess suPAR levels are those above 2.0 ng/ml, 2.1 ng/ml, 2.2 ng/ml, 2.3 ng/ml, 2.4 ng/ml, 2.5 ng/ml, 2.6 ng/ml, 2.7 ng/ml, 2.8 ng/ml, 2.9 ng/ml, 3.0 ng/ml, 3.1 ng/ml, 3.2 ng/ml, 3.3 ng/ml, 3.4 ng/ml, or 3.5 ng/ml. In some embodiments, treating the subject to reduce excess suPAR levels comprises administering an anti-suPAR therapy to the subject. In some embodiments, the anti-suPAR therapy is (1) administration of an anti-suPAR small molecule, peptide, or antibody treatment which specifically binds to suPAR, (2) administration of a small molecule, peptide, or antibody treatment which specifically binds to a ligand or receptor of suPAR, or (3) plasmapheresis to remove suPAR from the subject. In some embodiments, the anti-suPAR therapy inhibits the expression of suPAR. In some embodiments, the anti-suPAR therapy is a nucleic acid inhibitor of suPAR expression. In some embodiments, the nucleic acid inhibitor of suPAR expression is an antisense oligonucleotide (ASO), an siRNA, an shRNA, or an element of a Cas/CRISPR system. In some embodiments, the anti-suPAR therapy inhibits the activity of suPAR. In some embodiments, the anti-suPAR therapy is an antibody or antibody fragment that binds to suPAR or a ligand or receptor of suPAR and thereby inhibits the activity of suPAR. In some embodiments, the anti-suPAR therapy is a peptide or small molecule that binds to suPAR and thereby inhibits the activity of suPAR. In some embodiments, the small molecule is azeliragon

In some embodiments of the above-described methods, the sample is blood or urine, and the level of suPAR is determined or measured by immunoassay, immunoprecipitation, Western blot, flow cytometry, or protein microarray.

In some embodiments, the methods described herein find use in combination with methods for assessing and/or for preventing and/or treating cardiovascular diseases including, acute coronary syndrome, stable coronary artery disease, peripheral arterial disease, and congestive heart failure, with or without kidney disease or other methods, as described, for example, in U.S. Pub. No. 2020/0319196, which is hereby incorporated by reference in its entirety.

### BRIEF DESCRIPTION OF THE DRAWINGS

FIG. 1. Median coronary artery calcification (CAC) score at baseline and follow-up by suPAR categories. Median CAC score (Hounsfield units [HU]) based on Agaston scoring method at baseline and initial follow-up visits stratified by suPAR categories: 0-2.0 ng/mL, 2.0-2.5 ng/mL, 2.5-3.0 ng/mL, and >3.0 ng/mL. Error bars represent 95% confidence intervals. Abbreviations: CAC, coronary artery calcium; MESA, Multi-Ethnic Study of Atherosclerosis; suPAR, soluble urokinase plasminogen activator receptor.

FIG. 2. Cumulative incidence of any cardiovascular disease event by suPAR categories. Unadjusted Kaplan-Meier curves for the cumulative incidence of cardiovascular disease (CVD) events stratified by suPAR categories: 0-2.0 ng/mL (red), 2.0-2.5 ng/mL (green), 2.5-3.0 ng/mL (blue), >3 ng/mL (purple). The difference in cumulative incidence curves between suPAR categories was tested using the log-rank test. A CVD event was defined as the composite of myocardial infarction, resuscitated cardiac arrest, angina, revascularization, stroke (excluding transient ischemic attack), or death due to CVD.

FIG. 3. In-vitro and in-vivo expression of PLAUR missense variants and suPAR levels. Human suPAR levels in (A) the supernatant of human embryonic kidney (HEK) cells 48 hours after transfection with rs4760 and rs2302524 PLAUR variants, and in (B) C57BL/6j mice 24 hours after hydrodynamic tail vein injection of plasmid DNA containing wild-type (WT), the rs2302524 or rs4760 variant. *** indicates P<0.001 using the One-way ANOVA.

FIG. 4. Mendelian randomization phenome-wide association of genetically-predicted suPAR by rs4760 with CVD. Causal effect of suPAR on 13 cardiovascular diseases by Mendelian randomization using missense variant rs4760 as instrument. Effect estimates are provided per 1 standard deviation (SD) increase in suPAR levels. P values were adjusted using the false discovery rate method.

FIG. 5. SuPAR over-expression leads to increased atherosclerotic and necrotic plaques in a murine model of atherosclerosis. Wild-type (WT) and suPAR^{Tg} mice were maintained on a low-fat diet until 3-months of age and were then transfected with PCSK (proprotein convertase subtilisin/kexin)-9-adeno-associated virus (AAV) and fed a western diet (WD) for 10 wk. At this point, aortic roots were obtained, paraffin-embedded, and stained with H&E and Mac2 (galectin 3). Panel A, Cross sections of aortic roots from C57BL/6 WT and suPAR^{Tg} mice show total lesion area, outlined in dashed lines, and necrotic core area, outlined in dotted lines. Higher magnification shows the presence of necrotic core. Mac2 monoclonal antibody stain shown on aortic sinus cross sections from WT and suPAR^{Tg} mice. Scale bars: 100 μm and 50 μm. Panels B and C, Quantification of total lesion area and necrotic core area for all 30 sections. Panel D, Quantification of Mac2 staining as a percentage of total plaque area with necrotic area subtracted. 2-Way ANOVA for B and C and Student's t test for D. For atherosclerotic plaque and necrotic core area, n=18 WT and n=21 suPAR^{Tg} groups. Tissue sections are 6 μm each with 6 μm blank section between for a total 360 μm through the aortic sinus. Each data point represents a biological replicate for D. ** indicates P<0.01, **** indicates P<0.0001. suPAR: soluble urokinase plasminogen activator receptor, WT: wild-type.

FIG. 6. Association between baseline suPAR and incidence of any cardiovascular disease event.

FIG. 7. Distribution of suPAR levels by cohort. Violin and box plot for suPAR levels in each of the four main cohorts that are included in the overall meta-analysis. suPAR levels below 0.5 ng/mL and above 4.5 ng/mL were considered as outliers and excluded for the purposes of visualization.

FIG. 8. Manhattan plot for genome-wide associations with suPAR in multi-ancestry and European ancestry. Plot of the minus log 10 of the P-values observed in the genome-wide meta-analysis of suPAR levels in multi-ancestry (A) and European ancestry (B). Significant (P<5×10⁻⁸) signals were observed with variants in 8 genomic locations for both meta-analyses. The signal in the ACTR3C locus was specific to multi-ancestry analysis while the signal in MICA was specific to European ancestry analysis.

FIG. 9. UPAR protein sequence and missense variant annotation. Panel (A) The linear amino acid sequence of the full-length transcript was obtained from Ensembl. Domain annotations and uPA binding domains were annotated using previously published data.¹ (B) Representative protein diagram where approximate location of missense mutations on preprotein is indicated with rs number and arrow. Images were generated using BioRender.

FIG. 10A-H. Regional plots for loci associated with suPAR from European-ancestry analysis. Plots of the minus log 10 of the P-values observed in the genome-wide meta-analysis of suPAR levels in European ancestry of variants in each of the genome-wide significant loci. The horizontal dashed line was drawn at P=5×10−8. The different colors denote the correlation with the variant with the lowest P value in each locus. The regional plots were drawn using LocusZoom.

FIG. 11A-B. Regional plots in the PLAUR locus after sequential conditional analysis on top variants.

FIG. 12A-B. Cholesterol and suPAR levels in wild-type and transgenic mice prior to and 10 weeks after PSCK9-AAV transfection. A, Fasting total cholesterol was quantified from plasma via colorimetric assay. Baseline measure was from immediately prior to PCSK9-AAV transfection followed by 1 week to rest mice, and western diet feeding began at week 0. n=17 WT and n=23 suPAR-Tg mice per group. 2-Way ANOVA. B, Plasma suPAR level assessed by ELISA at baseline and at the end of the study. 2-Way ANOVA. ****=P<0.0001.

FIG. 13. QQ plots for genome-wide associations with suPAR. Quantile-Quantile plots for observed versus expected P-values in the multi-ancestry genome-wide meta-analysis (A) or European ancestry meta-analysis (B). The genomic control lambdas were 1.01 and 1.02 in the multi-ancestry and European ancestry analyses, respectively, indicating no evidence of population stratification.

FIG. 14. Chromatogram by Sanger sequencing of PLAUR reference allele and variants. The wild-type PLAUR (reference, Gene accession #NM_002659) was cloned into a pCMV6-entry vector (Origene). The PLAUR variants rs2302524 and rs4760 were created using the GeneArt site directed mutagenesis system (Thermo Scientific). Black arrow indicates reference nucleotide. Red arrow indicates variant nucleotide.

FIG. 15. SuPAR over-expression in mice leads to pro-atherosclerotic phenotype in circulating and aortic monocytes. Aortas and blood were harvested from disease-free C57BL/6 wild-type (WT) and suPAR over-expressing mice (suPARTg mice). Panel A, Aortas from WT (n=11) and suPARTg (n=11) mice were excised, cleaned of fat, and cultured for 24-hours. At this point, the conditioned culture medium was isolated and CCL2 level was assessed by enzyme linked immunosorbent assay (ELISA). Panel B, Aortas from WT (n=6) and suPARTg (n=6) mice were isolated, cleaned of fat, digested, stained with fluorescently labeled antibodies, and analyzed by flow cytometry. Quantification of F4/80-Ly-6G-CD11b+ monocytes from WT and suPARTg mice as a percentage of live Live CD11b+CD45+ cells and median fluorescent intensity (MFI) of CCR2 expression from WT on F4/80-Ly-6G-CD11b+ monocytes. Panel C, Blood from WT (n=) and suPARTg (n=) mice was isolated, red blood cells lysed, stained with fluorescently labeled antibodies, and analyzed by flow cytometry. MFI on Live CD45+CD11b+ monocytes for expression of CCR2, MHCII, and CX3CR1, and percentage of uPAR+ cells of live CD45+CD11b+ cells. For CCR2, WT n=16 and suPARTg n=15 and compared by Students T test. For MHCII, WT n=6 and suPARTg n=6. For CX3CR1, WT n=4 and suPARTg n=4. For uPAR+ cells, WT n=6 and suPARTg n=5. For MHCII, CX3CR1, and uPAR+ cells, Mann-Whitney U test. Panel D, Monocytes were isolated from spleens of WT and suPARTg mice and cultured in trans-well assays with either control cell culture media or cell culture media with CCL2 added. Quantification of fluorescence intensity of cellular dye was compared by 2-way ANOVA. n=6 for each group. Each data point represents a biological replicate. CCL2: C-C Motif Chemokine Ligand 2, CCR2: C-C Motif Chemokine Receptor 2, CX3CR1: C-X3-C Motif Chemokine Receptor 1, MHCII: major histocompatibility complex 2, suPAR: soluble urokinase plasminogen activator receptor, WT: wild-type.

### DEFINITIONS

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular compositions, methodologies, or protocols described herein, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein, the terms “administration” and “administering” refer to the act of giving a drug, prodrug, therapeutic, or other agent to a subject or in vivo, in vitro, or ex vivo cells, tissues, and organs. Exemplary routes of administration to the human body can be through space under the arachnoid membrane of the brain or spinal cord (intrathecal), the eyes (ophthalmic), mouth (oral), skin (topical or transdermal), nose (nasal), lungs (inhalant), oral mucosa (buccal), ear, rectal, vaginal, by injection (e.g., intravenously, subcutaneously, intratumorally, intraperitoneally, etc.) and the like.

The terms “biomarker” and “biological marker” are used synonymously herein and refer to a defined characteristic that is measured as an indicator of normal biological processes, pathogenic processes, or responses to an exposure or intervention, including therapeutic interventions. In some embodiments, a biomarker may comprise a substance whose detection indicates a particular disease state (e.g., the presence of an antibody may indicate an infection). More specifically, a biomarker may indicate a change in expression or state of a protein that correlates with the risk or progression of a disease, or with the susceptibility of the disease to a particular treatment. The types of substances that may be measured as biomarkers range widely and include, but are not limited to, molecular biomarkers (e.g., nucleic acids, gene products, and proteins), physiologic biomarkers (e.g., blood pressure or blood flow), or anatomic biomarkers (e.g., the structure of a particular organ).

As used herein, the term “diagnosis” encompasses determining the nature of disease in a subject, as well as determining the severity and probable outcome of disease or episode of disease and/or prospect of recovery (prognosis). “Diagnosis” can also encompass diagnosis in the context of rational therapy, in which the diagnosis guides therapy, including initial selection of therapy, modification of therapy (e.g., adjustment of dose and/or dosage regimen or lifestyle change recommendations), and the like.

The terms “individual,” “host,” “subject,” and “patient” are used interchangeably herein and refer to any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgus monkey, chimpanzee, etc.) and a human). Preferably, the subject is a human. The terms “individual,” “host,” “subject,” and “patient” are used herein irrespective of whether the subject has or is currently undergoing any form of treatment.

The terms “increased,” “increase,” and “elevated” may be used interchangeably herein and refer to an amount or a concentration in a sample that is higher or greater than a predetermined level or range, such as a typical or normal level found in a control group or control sample, or is higher or greater than another reference level or range (e.g., earlier or baseline sample). The terms “decreased,” “decrease,” “lowered,” and “reduced” may be used interchangeably herein and refer to an amount or a concentration in a test sample that is lower or less than a predetermined level or range, such as a typical or normal level found in a control group or control sample, or is lower or less than another reference level or range (e.g., earlier or baseline sample). The term “altered” refers to an amount or a concentration in a sample that is altered (increased or decreased) over a predetermined level or range, such as a typical or normal level found in a control group or control sample, or over another reference level or range (e.g., earlier or baseline sample).

The terms “prevent,” “prevention,” “prophylactic,” and the like refer to completely or partially inhibiting onset of a disease or symptom thereof. A “prophylactically effective amount” of a particular compound, drug, or agent refers to an amount effective, at dosages and for periods of time necessary, to achieve a desired prophylactic result (e.g., prevention of disease onset). The likelihood of developing a condition need not be completely eliminated to constitute prevention. If a composition or method step reduces the likelihood of developing a condition across a population, then the composition or method step prevents the condition within the scope herein.

As used herein, the terms “treatment,” “treating,” and the like refer to obtaining a desired pharmacologic and/or physiologic effect. Preferably, the effect is therapeutic, i.e., the effect partially or completely cures a disease and/or adverse symptom attributable to the disease. A “therapeutically effective amount” of a particular compound, drug, or agent refers to an amount effective, at dosages and for periods of time necessary, to achieve a desired therapeutic result. A therapeutically effective amount may vary according to factors such as the disease state, age, sex, and weight of a subject.

### DETAILED DESCRIPTION

Provided herein are methods for preventing or treating atherosclerosis by reducing levels or inhibiting the activity of soluble urokinase plasminogen activator receptor (suPAR) protein and/or and downstream effectors thereof (e.g., αvβ3 integrin, vitronectin, uPA, etc.) in a subject.

Experiments conducted during development of embodiments herein provide compelling epidemiologic, genetic and experimental evidence of a causal role for suPAR in atherosclerosis. In a multi-ethnic cohort of over 5000 participants without known CVD, high suPAR levels were found to be strongly associated with incident CVD and accelerated atherosclerosis as measured by CAC scores independently of established risk factors. In genetic analyses, two independent common missense variants were identified in PLAUR associated with higher plasma suPAR levels. One variant (rs4760, p.Leu317Pro) was confirmed experimentally in-vitro and in-vivo to lead to higher suPAR levels. Through Mendelian randomization, it was found that genetically predicted suPAR levels were causally linked to atherosclerotic phenotypes in the UK Biobank, notably coronary artery disease, myocardial infarction, and peripheral arterial disease. Additionally, rare, damaging variants of PLAUR were associated with lower risk of ischemic disease. Lastly, over-expression of suPAR in a murine model of atherosclerosis using Pcsk9-AAV coupled with western diet led to a dramatic increase in atherosclerotic plaque size with large necrotic cores and macrophage infiltration in suPAR mice compared to wild-type mice. These findings dovetail extensive experimental and clinical data on suPAR's role in kidney disease and characterize high suPAR levels as a shared risk factor and potential therapeutic target for CVD and chronic kidney disease (CKD).

Systemic inflammation is recognized as a key process common to CVD and CKD (Refs. 41-43; incorporated by reference in their entireties). The uPAR system is an important regulator of that process, notably through modulation of immune cell motility and extracellular matrix remodeling (refs. 3-9; incorporated by reference in their entireties). SuPAR levels are strongly induced by shared risk factors for CKD and CVD such as smoking, hypertension and diabetes mellitus, (Refs. 14, 19, 25, 44; incorporated by reference in their entireties) associated with coronary and peripheral atherosclerotic disease (refs. 27, 29, 32, 45-47; incorporated by reference in their entireties), and are predictive of incident kidney disease and CVD outcomes across age, gender, race and clinical settings, independently of the aforementioned risk factors (Refs. 11, 12, 14-16, 18, 19, 28, 30-39; incorporated by reference in their entireties). Accordingly, suPAR has been traditionally thought of as a biomarker of CVD and CKD. To determine whether high suPAR levels precedes CVD, MESA was leveraged—a cohort in which clinical CVD was an exclusion criterion—and it was found that high suPAR levels at baseline predicted accelerated atherosclerosis as measured by CAC and incident CVD events even in participants with CAC=0 and normal kidney function. Other biomarkers of inflammation have not exhibited a similar relationship with CAC (Refs. 48, 49; incorporated by reference in their entireties) which has prompted us to further explore suPAR's singular role in atherosclerosis—now supported by our genetic and experimental analyses.

Genome-wide association studies have revealed connections between common genetic variants and the risk for complex disease traits and quantitative traits such as the variation of plasma protein concentrations (Refs. 50-51; incorporated by reference in their entireties). These genetic variants, which are inherited independent of other disease risk modifiers, can be used in MR studies to determine if a specific protein plays a causal role in a complex disease (Refs. 52-54; incorporated by reference in their entireties). It was found that both common coding variation in the PLAUR gene shown experimentally to affect suPAR levels and rare coding predicted to damage uPAR/suPAR were linked to atherosclerotic disease. The genetic associations are thus unlikely biased due to pleiotropic effects on other genes and their impact on atherosclerosis have a very high likelihood of being mediated by suPAR. The rs2302524 variant did not lead to an increase in levels when expressed experimentally, and was not found to be linked to CVD phenotypes in a previous study (ref. 55; incorporated by reference in its entirety). The resulting amino-acid change encoded by rs2302524 (p.Lys220Arg) is located in the DIII domain of suPAR and results in an altered structure which may have a prolonged plasma half-life, increased binding affinity with the antibodies used in the immunoassay, or impaired downstream signaling. The rs4760 variant specifically increases suPAR levels without altering the structure of the circulating protein, as the variant is located only in a proprotein form of uPAR, suggesting that full-length (DI-DII-DIII) suPAR is the pathogenic form.

To confirm whether high levels of full-length suPAR accelerates atherosclerosis, a murine Pcsk9-AAV model of atherosclerosis was used, which allows for the study of immuno-metabolic processes without the confounding effects of germline alterations seen with the apolipoprotein E knockout and low-density lipoprotein receptor knockout models (Refs. 56, 57; incorporated by reference in their entireties). It was found that overexpression of suPAR led to a 2-fold increase in total atherosclerotic plaque size, 3.8-fold increase in necrotic core size, and a 2-fold increase in lesional macrophage infiltration in suPARTg compared to wild-type, without differences in lipid levels and regardless of kidney dysfunction. Extensive experimental evidence support a causal role for suPAR in kidney disease through its impact on both podocytes (Refs. 8, 10, 20, 21; incorporated by reference in their entireties) and proximal tubules (Ref. 18; incorporated by reference in its entirety), at least partially through binding and pathologic activation of αvβ3 integrin expressed on podocytes (Refs. 8, 10, 21, 58; incorporated by reference in their entireties). SuPAR's role in atherosclerosis may also be related to its binding of integrins (Ref. 59; incorporated by reference in its entirety). Integrins, notably αvβ3, are crucial in initiation of atherosclerosis in endothelial cells and promote inflammation through the NFκB pathway (Refs. 59-61; incorporated by reference in their entireties). Activation of integrins can also facilitate immune cell homing to the aorta and vascular remodeling (Ref. 62; incorporated by reference in its entirety).

Experiments were conducted during development of embodiments herein to provide a comprehensive approach in identifying a role for suPAR in atherosclerosis, involving epidemiologic and genetic analysis using large, well-established cohorts, and experiments using a murine model of atherosclerosis that does not involve germline alterations. Targeting inflammation as a strategy to decrease the risk of CVD has been shown to be a viable in recent trials using monoclonal antibodies to interleukin-10 and interleukin-6 (Refs. 43, 70, 71; incorporated by reference in their entireties). SuPAR has been targeted successfully in experimental models: bone marrow ablation (ref. 10; incorporated by reference in its entirety), monoclonal antibodies directed to suPAR (Ref. 8, 18; incorporated by reference in their entireties), or small molecule inhibitors of suPAR can prevent or reverse kidney injury (Ref. 24; incorporated by reference in its entirety). In patients with focal segmental glomerulosclerosis, plasmapheresis reduces suPAR levels, decreases β3-integrin activity, and stabilizes the disease (Refs. 72-74; incorporated by reference in their entireties).

The present disclosure is predicated, at least in part, on the discovery that the levels of soluble urokinase plasminogen activator receptor (suPAR) in blood are not only predictive of atherosclerosis, but that increased suPAR levels contributes to and is a causative factor in the development of atherosclerotic plaque. The experiments conducted during development of embodiments herein demonstrate that treatments that reduce suPAR levels and/or inhibit suPAR activity in a subject (e.g., in the blood) are useful for the treatment and/or prevention of atherosclerosis. In some embodiments, atherosclerosis is treated or prevented by directly inhibiting expression or activity of suPAR. In other embodiments, atherosclerosis is treated or prevented by inhibiting the expression or activity of one or more downstream effectors of suPAR, such as αvβ3 integrin, vitronectin, uPA, etc.

Soluble urokinase plasminogen activator receptor (suPAR) (NCBI Accession No. AAK31795) is the circulating form of a glycosyl-phosphatidylinositol-anchored three-domain membrane protein that is expressed on a variety of cells, including immunologically active cells, endothelial cells, podocytes, keratinocytes, fibroblasts, smooth muscle cells, megakaryocytes, and certain tumor cells (Thuno et al., Disease Markers, 27: 157-172 (2009); Wei et al., Nat Med, 14: 55-63 (2008); and Huai et al., Science, 311: 656-659 (2006); incorporated by reference in its entirety). Both the circulating and membrane-bound forms of suPAR are directly involved in the regulation of cell adhesion and migration through binding of integrins (Thuno et al., supra). The circulating form is produced by cleavage of membrane-bound urokinase-type plasminogen activator receptor and is readily detected in plasma, serum, urine, and other bodily fluids. Elevated suPAR levels have been associated with poor outcomes in various patient populations (see, e.g., Theilade et al., J Intern Med, 277: 362-371 (2015); Yoo et al., J Am Soc Nephrol, 26: 133-147 (2015); de Bock CE and Wang Y., Med Res Rev, 24: 13-39 (2004); Backes et al., Intensive Care Med, 38: 1418-1428 (2012); Borne et al., Eur J Heart Fail, 16: 377-383 (2014); Eugen-Olsen et al., J Intern Med, 268: 296-308 (2010); Fuhrman B., Atherosclerosis, 222: 8-14 (2012); Lyngbok et al., Int J Cardiol, 167: 2904-2911 (2013); and Eapen et al., J Am Heart Assoc, 3: e001118-e001118 (2014); incorporated by reference in their entireties). However, prior studies have not demonstrated that increased suPAR levels contribute to atherosclerosis, nor that reduction of suPAR provides treatment/prevention of atherosclerosis. Increased activation of the immune system leads to increased serum suPAR levels, which has been documented in several pathological conditions, including paroxysmal nocturnal hemoglobinuria, human immunodeficiency virus type 1 (HIV-1) infection, malaria, pneumococcal and Streptococcus pneumonia bacteraemia, sepsis, bacterial and viral CNS infection, active tuberculosis (TB) and also in various types of solid tumors (e.g., non-small cell lung cancer, breast, colorectal, prostate, and ovarian cancer) (Thuno et al., supra). suPAR also has been implicated in the pathogenesis of kidney disease, specifically focal segmental glomerulosclerosis and diabetic nephropathy, through interference with podocyte migration and apoptosis (Hayek et al., N Engl J Med, 373: 1916-1925 (2015); incorporated by reference in its entirety). Furthermore, high blood concentrations of suPAR independently predict high mortality in both patients and healthy individuals (Eugen-Olsen et al., Int J Tuberc Lung Dis, 6: 686-692 (2002); incorporated by reference in its entirety).

suPAR is well studied in kidney disease. Interfering with the suPAR pathway by bone marrow ablation (removing the cells that produce suPAR in highest quantities), monoclonal antibodies directed to suPAR, or small molecule inhibitors of suPAR, can prevent and reverse kidney injury in experimental models. In patients with focal segmental glomerulosclerosis, non-selective plasmapheresis reduces suPAR levels, decreases β3-integrin activity, and stabilizes the disease. SuPAR signaling is blocked by the RAGE antagonist azeliragon, and by RAGE knockdown by siRNA methods.

Pathologic integrin activation by suPAR induces podocyte cytoskeletal restructuring, autophagy, and effacement leading to proteinuria and chronic kidney dysfunction. A recent study examined the initial transduction steps used by suPAR in cultured mouse podocytes. The receptor for advanced glycation end-products (RAGE) co-immunoprecipitates with αV and β3 integrin subunits, which have been previously shown to initiate suPAR signal transduction at the podocyte cell surface. In the earliest step of suPAR signaling, RAGE functions as an essential co-receptor for suPAR signaling in podocytes.

RAGE is a membrane bound multiligand pattern recognition receptor that under basal conditions is predominantly expressed in the lung on the basolateral surface of alveolar type 1 cells. RAGE plays a critical role in lung maturation and function. Two C-truncated soluble forms of RAGE, sRAGE and esRAGE circulate in the blood and other biological fluids, acting as endogenous competitive RAGE decoys preventing responses mediated by RAGE activation. RAGE has been demonstrated as a key molecule driving inflammatory disease states. Advanced glycation end products (AGEs), high mobility group box protein-1 (HMGB1) and S100 proteins are the major RAGE ligands and potent proinflammatory molecules. RAGE activation after ligand binding increases receptor expression and triggers multiple proinflammatory and procoagulant pathways [e.g. nuclear factor-κB (NFκB), Akt, p38, and MAP kinases].

SuPAR plays an important role in the recruitment of monocytes to inflamed tissue, where complexes of uPAR and aMb2 integrin/Mac-1 expressed in leukocytes interact with intracellular Src kinases upon binding to vitronectin or fibrinogen, thereby regulating adhesion and cell migration of mononuclear cells. Full-length suPARI-III is able to bind vitronectin, to form a uPA-suPAR-vitronectin complex, which may allow vitronectin-directed activation of uPA at cellular surfaces or extracellular matrix sites. Second, suPARII-III may directly exert multiple pro-inflammatory functions by exposing an N-terminal SRSRY amino acid sequence (SEQ ID NO: 1). This SRSRY sequence acts as a chemotactic agent by interacting with the G protein-coupled receptor FPR-like receptor 1 (FPRL1) expressed on immune cells, including monocytes, lymphocytes, and neutrophils.

Thus, uPAR, uPA, and b2 integrin provide the adhesion/degradation interactions between immune cells and endothelial cells or extracellular matrix, required for leukocytes to invade inflamed tissue in response to a chemotactic signal. Additional mechanisms by which uPAR regulates inflammatory processes have been suggested. These include co-localization of uPAR with cytokeratin-1 (CK1) and globular C1q receptor (gC1qR) on the surface of endothelial cells, which promotes release of the vasodilator bradykinin. Another mechanism is the simultaneous stimulation of uPAR, b2 integrin, and gC1qR by cleaved high molecular weight kininogen, which induces release of cytokines (IL-1b, IL-6) and chemokines (IL-8, monocyte chemoattractant protein-1 [MCP-1]) from blood mononuclear cells.

In some embodiments, the methods described herein involve measuring and determining the level of suPAR protein in a sample obtained from a subject. The terms “sample” and “biological sample” are used interchangeably herein and refer to bodily fluids such as blood-related samples (e.g., whole blood, serum, plasma, and other blood-derived samples), urine, cerebral spinal fluid, bronchoalveolar lavage, and the like. Another example of a sample is a tissue sample. A biological sample may be fresh or stored (e.g., blood or blood fraction stored in a blood bank). The biological sample may be a bodily fluid expressly obtained for the methods described herein or a bodily fluid obtained for another purpose which can be sub-sampled for the methods of this disclosure. In certain embodiments, the biological sample is whole blood. Whole blood may be obtained from the subject using standard clinical procedures. In other embodiments, the biological sample is plasma. Plasma may be obtained from whole blood samples by centrifugation of anti-coagulated blood, which provides a buffy coat of white cell components and a supernatant of the plasma. In certain embodiments, the biological sample may be serum. Serum may be obtained by centrifugation of whole blood samples that have been collected in tubes that are free of anti-coagulant. The blood is permitted to clot prior to centrifugation. The yellowish-reddish fluid that is obtained by centrifugation is the serum. In another embodiment, the sample may be urine. The sample may be pretreated as necessary by dilution in an appropriate buffer solution, heparinized, concentrated if desired, or fractionated by any number of methods, including but not limited to ultracentrifugation, fractionation by fast performance liquid chromatography (FPLC), or precipitation of apolipoprotein B containing-proteins with dextran sulfate or other methods. Any of a number of standard aqueous buffer solutions at physiological pH, such as phosphate, Tris, or the like, can be used. Methods well-known in the art for collecting, handling and processing urine, blood, serum and plasma, and other body fluids, may be used in the practice of the present disclosure.

In embodiments herein, the level of suPAR protein may be measured, determined, and/or quantified using any suitable method for protein detection and/or quantification known in the art. Such methods include, but are not limited to, immunoassays (e.g., enzyme linked-immunosorbent assay (ELISA), protein immunoprecipitation, immunoelectrophoresis, chemical analysis, SDS-PAGE and Western blot analysis, protein immunostaining, electrophoresis analysis, competitive binding assays, functional protein assays, protein microarray, or chromatography or spectrometry methods (e.g., high-performance liquid chromatography (HPLC), mass spectrometry, liquid chromatography-mass spectrometry (LC/MS), capillary electrophoresis (CE)-MS, or any separating front end coupled with MS detection and quantification) (see, e.g., Salvatore Sechi, Quantitative Proteomics by Mass Spectrometry (Methods in Molecular Biology) 2nd ed. 2016 Edition, Humana Press (New York, NY, 2009); Daniel Martins-de-Souza, Shotgun Proteomics: Methods and Protocols 2014 edition, Humana Press (New York, NY, 2014); Jörg Reinders and Albert Sickmann, Proteomics: Methods and Protocols (Methods in Molecular Biology) 2009 edition, Humana Press (New York, NY, 2009); and Jörg Reinders, Proteomics in Systems Biology: Methods and Protocols (Methods in Molecular Biology) 1st ed. 2016 edition, Humana Press (New York, NY, 2009); incorporated by reference in their entireties).

In some embodiments, suPAR may be present in the sample at low levels that may not be efficiently detected using conventional methods. In such cases, the suPAR protein may be detected using ultrasensitive methodologies and devices specifically designed for detecting low abundant proteins in a sample. Examples of such methodologies and devices include, but are not limited to, microfluidic analytical systems (such as those described in, e.g., Martel, J. M. and Toner, M., Annu Rev Biomed Eng., 16: 371-96 (2014); Martel et al., Annu Rev Biomed Eng., 16: 371-96 (2014); Malhotra et al., Anal. Chem., 84, 6249-6255 (2012); and U.S. Patent Application Publication 2018/0161775 A1), ultra-sensitive ELISA assays (see, e.g., Schubert et al., Scientific Reports, 5: Article number: 11034 (2015)), and nanoparticle-based systems (see, e.g., Li et al., Biosensors and Bioelectronics, 68: 626-632 (2015); incorporated by reference in their entireties).

Methods and devices for protein detection and quantification are further described in, e.g., Powers, A. D, and S. P. Palecek, Journal of Healthcare Engineering, 3(4): 503-534 (2012); incorporated by reference in its entirety, and are available from a variety of commercial sources, any of which may be used in the methods described herein.

In certain embodiments, the methods described herein involves comparing the levels of suPAR in a patient sample with a predetermined value or cutoff. The terms “predetermined cutoff,” “cutoff,” “predetermined value,” “reference level,” and “threshold level,” as used herein, refer to an assay cutoff value that is used to assess diagnostic, prognostic, or therapeutic efficacy results by comparing the assay results against the predetermined cutoff/level, where the predetermined cutoff/value already has been linked or associated with various clinical parameters (e.g., presence of disease, stage of disease, severity of disease, progression, non-progression, improvement of disease, etc.). The disclosure provides exemplary predetermined levels and reference levels. However, it is well-known that cutoff values may vary depending on the nature of the detection method or assay. Whereas the precise value of the predetermined cutoff/value may vary between assays, the correlations as described herein should be generally applicable.

The predetermined value can take a variety of forms. For example, the predetermined value can be single cut-off value, such as a median or mean. The predetermined value can be established based upon comparative groups, such as where the risk of coronary artery disease in one defined group is double the risk in another defined group. In yet another alternative, the predetermined value can be a range, for example, where the tested population is divided equally (or unequally) into groups, such as-a low-risk group, a medium-risk group, and a high-risk group, or into quadrants, the lowest quadrant being individuals with the lowest risk and the highest quadrant being individuals with the highest risk.

The predetermined value can depend upon the particular population selected. For example, an apparently healthy population will have a different normal range of biomarker expression levels than will a population comprised of patients with symptoms of cardiovascular disease or heart failure. In another embodiment, a population comprised of patients with congestive heart failure will have a different range of suPAR expression levels than will a population of patients with stable cardiovascular disease. Accordingly, the predetermined values selected may take into account the disease category in which an individual is grouped. Appropriate ranges and categories can be selected by those of ordinary skill in the art using routine methods. In some embodiments, an algorithm may be used to determine a predetermined value or threshold for decision making. Such an algorithm may consider a variety of factors, including, for example, (i) the age of the subject (e.g., higher threshold at higher age), (ii) renal function (e.g., lower threshold with better renal function because the kidneys are actively clearing suPAR), and (iii) gender (e.g., about 3 ng/mL suPAR (male) and about 4 ng/mL suPAR (female)).

It will be appreciated that a “normal” or “baseline” level of suPAR protein in a sample obtained from a subject (e.g., a human subject) will vary depending on a variety of factors, such as the subject's gender, age, overall health, the sample type (e.g., whole blood, serum, etc.), the suPAR isoform measured, and the assay used for measuring suPAR levels. Generally, a normal level of suPAR in human blood (or blood fraction) is less than 2.5 ng/mL (e.g., about 0.5 ng/mL, about 1.0 ng/mL, about 1.5 ng/mL, about 2.0 ng/mL, about 2.2 ng/mL, or about 2.4 ng/mL). In some embodiments, an elevated level of suPAR in human blood is greater than 2.5 ng/mL (e.g., 2.6 ng/mL, 2.7 ng/mL, 2.8 ng/mL, 2.9 ng/mL, 3.0 ng/mL, 3.1 ng/mL, 3.2 ng/mL, 3.3 ng/mL, 3.4 ng/mL, 3.5 ng/mL, 4.0 ng/mL, 4.5 ng/mL, 5.0 ng/mL, 6.0 ng/mL, 7.0 ng/mL, 8.0 ng/mL, 9.0 ng/mL, or greater, or ranges therebetween) (see, e.g., Rabna et al., PLoS One, 7(8): e43933 (2012); Lawn et al., BMC Infect Dis., 7: 41 (2007); and Schneider et al., BMC Infectious Diseases, 7: 134 (2007)). It will be appreciated that the normal level of suPAR in men is less than about 2.1 ng/mL and in women is less than about 2.5 ng/mL. A suPAR level that is higher than the normal range may be indicative of CVD, CKD or a elevated risk thereof.

The level of suPAR protein may be considered “elevated” if the level measured is above a predetermined threshold level. In one embodiment, such a threshold level can be set to the 90th-percentile or to the 95th-percentile of a healthy control population. Preferably, the threshold level is established at the 95th-percentile of a healthy control population. In some embodiments, a particular therapeutic decision or risk assessment for a subject suffering from, or at risk of suffering from, atherosclerosis is indicated when the level of suPAR is, for example, at least 2-fold greater (e.g., 2, 5, 10, 15, 20, 25, 30, 35, 40, 45, 50-fold, or greater) than a predetermined normal level of suPAR. In some embodiments, excess suPAR levels (e.g., levels placing a subject at risk for atherosclerosis) are those above 2.0 ng/ml, 2.1 ng/ml, 2.2 ng/ml, 2.3 ng/ml, 2.4 ng/ml, 2.5 ng/ml, 2.6 ng/ml, 2.7 ng/ml, 2.8 ng/ml, 2.9 ng/ml, 3.0 ng/ml, 3.1 ng/ml, 3.2 ng/ml, 3.3 ng/ml, 3.4 ng/ml, or 3.5 ng/ml.

In some embodiments, the methods of assessing atherosclerosis by measuring levels of suPAR are combined with other diagnostic methods for assessing atherosclerosis, such as blood tests (e.g., blood sugar, cholesterol, C-reactive protein, etc.), electrocardiogram, exercise stress test, echocardiogram, doppler ultrasound, ankle-brachial index test, cardiac catheterization and angiogram, coronary calcium scan, imaging (e.g., magnetic resonance angiography (MRA) or positron emission tomography (PET), etc.).

In some embodiments, methods are provided herein for the treatment or prevention of atherosclerosis and/or CVD. In particular, methods are provided for reducing blood suPAR levels, inhibiting the activity of suPAR, or inhibiting the expression or activity of a downstream effector of suPAR (e.g., αvβ3 integrin, vitronectin, uPA) in a subject to treat or prevent atherosclerosis and/or CVD.

In some embodiments, an agent is administered to a subject in need thereof that modulates (e.g., inhibits) expression and/or activity of suPAR or a downstream effector of suPAR (e.g., αvβ3 integrin, vitronectin, uPA) in the subject. The agent can be any agent that modulates expression of suPAR, urokinase receptor molecules, or downstream effectors of suPAR, such as for example, antisense oligonucleotides, antibodies, small molecules (e.g., azeliragon), and the like. In some embodiments, the agent modulates or inhibits suPAR (or a downstream effector of suPAR) expression, function and/or activity by about 5% as compared to a normal control, preferably by about 10%, preferably by about 50%, preferably by about 80%, 90%, 100%. In some embodiments, an agent binds to suPAR (or a downstream effector of suPAR) and blocks or reduces its activity. In some embodiments, an agent binds to a ligand or receptor of suPAR and blocks or reduces suPAR activity through that binding. In some embodiments, an agent modulates the degradation and/or rate of degradation of suPAR. In some embodiments, agents which modulate suPAR activity and/or expression comprise oligonucleotides, polynucleotides, peptides, polypeptides, antibodies, aptamers, small molecules, organic molecules, inorganic molecules or combinations thereof.

Some embodiments relate to targeted binding agents that bind suPAR (or a downstream effector of suPAR) and affect the activity thereof. Examples include, monoclonal antibodies that bind suPAR and affect suPAR function (or a downstream effector of suPAR). Other embodiments relate to anti-suPAR antibodies with high binding affinity for suPAR, the ability to neutralize suPAR in vitro and/or in vivo, and the ability to inhibit suPAR expression and/or function. In another embodiment, the invention relates to fully human anti-suPAR antibodies with desirable properties from a therapeutic perspective, including high binding affinity for suPAR, the ability to neutralize suPAR in vitro and/or in vivo.

In certain embodiments, the invention includes antibodies that bind to suPAR (or a downstream effector of suPAR) with very high affinities (Kd). For example a human, rabbit, mouse, chimeric or humanized antibody that is capable of binding suPAR with a Kd less than, but not limited to, 10⁻¹, 10⁻⁶, 10⁻⁷, 10⁻⁸, 10⁻⁹, 10⁻¹⁰, or 10⁻¹¹ M, or any range or value therein. Affinity and/or avidity measurements can be measured by KinExA™ and/or BIACORE™.

Some embodiments include isolated antibodies, or fragments of those antibodies, that bind to suPAR (or a downstream effector of suPAR). As known in the art, the antibodies can be, for example, polyclonal, oligoclonal, monoclonal, chimeric, humanized, and/or fully human antibodies. Embodiments of the invention described herein also provide cells for producing these antibodies. It will be appreciated that embodiments of the invention are not limited to any particular form of an antibody or method of generation or production. For example, the anti-suPAR antibodies herein may be a full-length antibody (e.g., having an intact human Fc region) or an antibody fragment (e.g., a Fab, Fab′, F(ab′)₂, Fv or Dab (Dabs are the smallest functional binding units of human antibodies). In addition, the antibody may be manufactured from a hybridoma that secretes the antibody, or from a recombinantly produced cell that has been transformed or transfected with a gene or genes encoding the antibody.

Other embodiments of the invention include isolated nucleic acid molecules encoding any of the targeted binding agents, antibodies or fragments thereof as described herein, vectors having isolated nucleic acid molecules encoding anti-suPAR antibodies or a host cell transformed with any of such nucleic acid molecules. In addition, one embodiment of the invention is a method of producing an anti-suPAR antibody of the invention by culturing host cells under conditions wherein a nucleic acid molecule is expressed to produce the antibody followed by recovering the antibody. It should be realized that embodiments of the invention also include any nucleic acid molecule which encodes an antibody or fragment of an antibody of the invention including nucleic acid sequences optimized for increasing yields of antibodies or fragments thereof when transfected into host cells for antibody production.

In some embodiments, a nucleic acid is administered to a subject that inhibits expression of suPAR. Nucleic acid-based agents such as antisense molecules and ribozymes can be utilized to target both the introns and exons of the suPAR genes as well as at the RNA level to inhibit gene expression thereof, thereby inhibiting the activity of the targeted suPAR. Further, triple helix molecules may also be utilized in inhibiting the suPAR gene activity. Techniques for the production and use of such molecules are well known to those of skill in the art, and are succinctly described below.

In some embodiments, a small molecule inhibitor of suPAR (or a downstream effector of suPAR) is administered to reduce suPAR activity in the subject.

In some embodiments, plasmapheresis is conducted on blood from a subject to reduce blood suPAR levels. Plasmapheresis refers to a broad range of procedures in which extracorporeal separation of blood components results in a filtered plasma product. In some embodiments, a portion of plasma is removed from a subject, treated to remove all or a portion of the suPAR present in the removed plasma, and the plasma is returned to the subject. Plasmapheresis may be performed by continuous flow centrifugation, plasma filtration, contact with suPAR-binding agents, etc.

### EXPERIMENTAL

The following further illustrate the invention but, of course, should not be construed as in any way limiting its scope.

### Epidemiologic, Genetic and Experimental Evidence of Soluble Urokinase Plasminogen Activator Receptor's (SuPAR) Pathogenic Role in Atherosclerosis

### Materials and Methods

### Overall Study Design

It was first assessed whether suPAR levels in humans were predictive of accelerated atherosclerosis and CVD outcomes. To that end a large multiethnic cohort of over 5000 participants was leveraged, in whom suPAR levels were measured and who underwent serial assessment of coronary artery calcium (CAC)—a surrogate of atherosclerotic burden. Evidence of a causal role for suPAR in atherosclerosis was then identified through a genome-wide study of suPAR levels was performed in 12,000 individuals, followed by experimental confirmation of the impact of identified missense variants on suPAR levels, and finally a Mendelian randomization study linking genetically determined suPAR levels to CVD phenotypes. Lastly, a murine experimental model was used to assess whether suPAR over-expression led to a higher burden of atherosclerosis.

### SuPAR, Coronary Calcification and Incident Cardiovascular Events

### Study Cohort

The Multi-Ethnic Study of Atherosclerosis (MESA) is a multicenter observational cohort which purpose is to identify risk factors for the incidence and progression of cardiovascular disease (CVD). A detailed description of the study design and methods have been published previously (Ref. 75; incorporated by reference in its entirety).⁷⁵ In summary, 6,814 (3,601 women; 3,213 men) participants aged 45-84 years who identified as either White, Black, Hispanic, or Chinese were enrolled between 2000-2002 at 6 participating communities across the United States. Participants were eligible if they were free of clinical CVD at enrollment. All participants gave informed consent, and the study protocol was approved by the Institutional Review Board at each site. For the present study, all participants who provided plasma samples for suPAR biomarker measurements at enrollment were included (n=5,406).

### Measurement of Coronary Artery Calcium (CAC)

A detailed description of the methodology for the acquisition and interpretation of CAC scores in MESA have been published previously (Ref. 76; incorporated by reference in its entirety). Computed tomographic (CT) scanning of the chest was performed using either electron-beam CT (Ref. 77; incorporated by reference in its entirety) (Chicago, Los Angeles, and New York field center) or using a multidetector CT system (Ref. 78; incorporated by reference in its entirety) (Baltimore, Forsyth Country, and St. Paul field centers). CAC scores were calculated using the Agatston score and adjusted with a standard calcium phantom that was scanned with the participant (Ref. 79; incorporated by reference in its entirety). The mean Agatston score (Hounsfield units [HU]) was used in all analyses. Inter-observer and intra-observer agreement was high (k statistic=0.90 and 0.93, respectively). CAC scores were measured at baseline (Exam 1; July 2002-August 2002) with initial follow-up measurements performed on half of the cohort at Exam 2 (September 2002-January 2004) and the other half at Exam 3 (March 2004-July 2005). A quarter of participants were selected for CAC measurement at Exam 4 (September 2005-May 2007).

### Measurement of SuPAR

SuPAR was measured using a commercially available enzyme-linked immunosorbent assay (suPARnostic©, ViroGates, Copenhagen, Denmark). The lower limit of detection of the assay is 100 pg/mL. The inter-assay coefficient of variation determined using blinded replicate samples from participants ranged from 8-11% depending on the cohort. SuPAR levels are stable in stored plasma and serum samples, with levels reproducible in samples stored for over 5 years at −80° C. (Refs. 80-81; incorporated by reference in their entireties).

### SuPAR and CAC

Clinical characteristics for the cohort are reported stratified by suPAR categories (0-2.0 ng/mL, 2.0-2.5 ng/mL, 2.5-3.0 ng/mL, and >3.0 ng/mL). The correlation between suPAR and CAC scores at baseline was examined using Spearman Rank. To test whether suPAR levels (log-transformed base 2) are independently associated with CAC at baseline, linear regression was used with CAC as the dependent variable adjusted for CVD risk factors including age, sex, race, body-mass index, history of smoking, estimated glomerular filtration rate (eGFR) using the Chronic Kidney Disease Epidemiology Collaboration equation (Ref. 82; incorporated by reference in its entirety), low density lipoprotein levels (LDL), high density lipoprotein (HDL) levels, hypertension, and diabetes mellitus. The median CAC scores were then visualized at baseline and follow-up stratified by suPAR categories using bar graphs.

To determine whether suPAR levels at Exam 1 were associated with an increase in CAC over time, generalized estimating equations modeling with CAC as a continuous and longitudinal variable were generated and the interaction term suPAR*follow-up time was examined. The model was adjusted for the aforementioned variables in addition to baseline CAC.

### SuPAR and Cardiovascular Events

It was then assessed whether suPAR levels were predictive of CVD events. A CVD event was defined in MESA as the composite of myocardial infarction, resuscitated cardiac arrest, angina, revascularization, stroke (excluding transient ischemic attack), or death due to CVD (refs. 75-76; incorporated by reference in their entireties). Stepwise multivariable-adjusted Cox proportional hazards modeling was used to assess the contribution of relevant factors such as eGFR and CAC to the association between suPAR and CVD events. Model 0 (suPAR alone) was unadjusted; Model 1 was adjusted for age, sex, race, body-mass index, LDL, HDL, hypertension, and diabetes mellitus; Model 2 included all variables in Model 1 in addition to eGFR; and Model 3 included the variables in Model 2 with the addition of baseline CAC. SuPAR was modeled as a continuous (log-transformed base 2) and categorical variable (0-2.0 ng/mL, 2.0-2.5 ng/mL, 2.5-3.0 ng/mL, and >3.0 ng/mL) in all models. Follow-up time began at baseline until a CVD event, drop out, death, or end of the study period. Unadjusted Kaplan-Meier cumulative incidence curves for CVD events were generated for suPAR categories and compared using the log-rank test. A complete case analysis was performed. A two-sided P-value<0.05 was used to determine statistical significance. Analyses were performed using R Version 4.1.0 (R Foundation for Statistical Computing, Vienna, Austria).

### Genetic Determinants of SuPAR and the Link to Atherosclerosis

Plasma suPAR levels were measured using immunoassay in 4 different cohorts: the Trinity Student Study (TSS), the Genes and Blood-Clotting cohort (GABC), MESA and the Malmo Diet and Cancer Study (MDCS); totaling 12,937 participants. GWAS and meta-analysis were performed to identify genetic determinants of suPAR levels and replicated our findings in 12,177 healthy participants of the Danish Blood Donor Study (DBDS) where suPAR levels were measured using the same immunoassay. The top two significantly associated missense variants of PLAUR were then expressed in human embryonic kidney cells (HEK), and in C57BL/6j mice to determine which variants led to significant increases in suPAR levels. The UK Biobank was then leveraged to perform MR and assess for a causal link between genetically determined suPAR levels and CVD (n=408,894) (Ref. 83; incorporated by reference in its entirety).

### Genome-Wide Association Study Cohorts and Analysis

### Cohorts

The TSS is a cohort of 2,179 unrelated healthy and ethnically Irish individuals between 21 and 24 years old (59% women, all European ancestry) (Ref. 84; incorporated by reference in its entirety). The GABC cohort comprises 931 young and healthy students between 14-35 years of age (63% women, all European ancestry) (Ref. 85; incorporated by reference in its entirety). The MESA cohort included 5,092 unrelated participants aged 45-84 years (53% women, 38% European ancestry, 28% African-American, 22% Hispanic-American, and 11% Asian-American) free from CVD (Ref. 86; incorporated by reference in its entirety). The MDCS is a Swedish population-based cohort which included 4,735 randomly selected unrelated participants between 44-73 years of age (59% women, age range: all European ancestry) (Ref. 87; incorporated by reference in its entirety). Lastly, the DBDS Genomic cohort comprises a subset of 12,177 healthy blood donors aged 18-66 years (47% women, all European ancestry) (Ref. 88; incorporated by reference in its entirety).

Quality control measures were performed to exclude low quality samples and low quality variants within each study prior to imputation to reference genomes. In general, samples were excluded if they showed discordance between genetically inferred and reported sex, low call rate and duplications. Variants were excluded if they deviated from Hardy-Weinberg equilibrium. Imputation was done to predict non-genotyped variants. The TSS, GABC and MESA were imputed using TOPMed Freeze 5b (GCRh 38). The MDCS was imputed using the Haplotype Reference Consortium reference panel (GRCh 37) (Refs. 89-90; incorporated by reference in their entireties). The build was liftover to GRCh 38 using CrossMap (Ref. 91; incorporated by reference in its entirety). The DBDS was imputed using 1 KG phase 3, HapMap and a dataset consisting of >6,000 Danish whole-genome sequences.

### Genome-Wide Analysis

Genome-wide association analyses were performed with natural log suPAR levels adjusted for age, sex and the first 10 principal components of ancestry followed by inverse-normal transformation within each study and ancestry combination using array data imputed to reference genomes. Single-variant association analyses were performed using linear regression in PLINK v2.0 (Ref. 92; incorporated by reference in its entirety) within each study-ancestry combination. For GABC, linear mixed models incorporating a kinship matrix were performed using RVTESTS (ref. 93; incorporated by reference in its entirety). Overall, our analyses resulted in genome-wide summary data from European ancestry datasets from MDC (n=4,735), TSS (n=2,179), MESA (n=2,024) and GABC (n=931), and African—(n=1,363), East Asian—(n=623) and Hispanic—(n=1,082) from MESA. Quality control measures were performed on each of the summary association datasets prior to meta-analysis (Refs. 94-95; incorporated by reference in their entireties). Within each dataset, variants were filtered out that had minor allele count less than 20, Hardy-Weinberg equilibrium P value less than 5×10⁻⁶, low imputation quality (INFO<0.6), multi-allelic variants and palindromic variants (A/T or C/G) with minor allele frequency above 0.4.

### Meta-Analysis

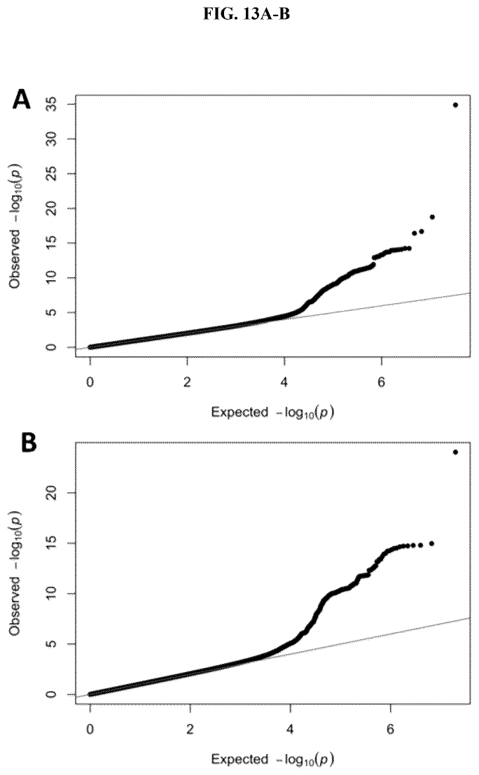

Multi-ancestry and European ancestry specific inverse-variance weighted fixed-effects meta-analyses was performed using METAL software (Ref. 94; incorporated by reference in its entirety). Quantile-quantile plots were generated to assess for genomic control and structure within our data (FIG. 13). To identify leading and independent variants from each meta-analysis, pruning and thresholding was performed using “clump” flag in PLINK. PLINK implements an iterative multistep process where variants are sorted by their P-values and those in linkage-disequilibrium are removed (r²<0.05 and within 250 kilobases from the lead variant). The process was repeated until the genome-wide significance threshold of 5×10⁻⁸ is reached. Top variants were defined as those with P-value<5×10⁻⁸ and are independent of each other. The identified variants in the DBDS cohort were then investigated. Functional annotations for top variants were obtained from Ensemble Variant Effect Predictor (Ref. 95; incorporated by reference in its entirety).

### In-Vitro and In-vivo Expression of PLAUR Missense Variants

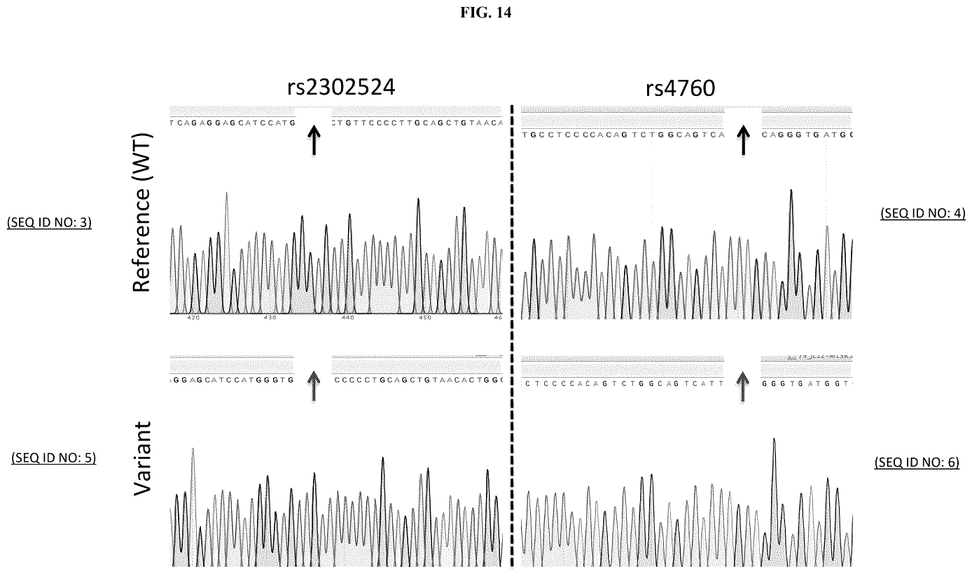

PLAUR variants rs2302524 and rs4760 (FIG. 14) were generated the using the GeneArt site-directed mutagenesis system (Thermo Scientific, Waltham, MA) and wild-type PLAUR (reference, Gene accession #NM_002659) cloned into a pCMV6-entry vector (Origene, Rockville, MD).

Equal amounts (12 μg) of plasmid DNA encoding vector control, human reference or the PLAUR missense variants, were transfected into HEK293T cells using the FuGene 6 transfection reagent (Promega, Madison, WI). The conditioned media and cells from each plate were harvested 48-hours post-transfection to measure suPAR in the supernatant using the Human uPAR Quantikine ELISA kit (R&D Systems, Minneapolis, MN).

Hydrodynamic tail-vein injection of plasmid DNA encoding reference human PLAUR (n=5), PLAUR variant rs2302524 (n=9), and PLAUR variant rs4760 (n=7) was performed in 8-week-old C57BL/6j female mice, and serum suPAR levels were measured 24 after injection using the Human uPAR Quantikine ELISA kit.

### Mendelian Randomization Analysis

The UK Biobank was leveraged for MR analysis in 408,894 participants of European-ancestry (UK Biobank Resource under Application Number 59206) (Ref. 96; incorporated by reference in its entirety). Details of measures for variant and sample quality control have been previously reported (Ref. 97; incorporated by reference in its entirety). The rs4760—the PLAUR missense variant confirmed to alter suPAR levels in both in-vitro and in-vivo models—was used as an instrument for MR analyses of 13 cardiovascular phenotypes from the UK Biobank (Ref. 98; incorporated by reference in its entirety). Wald ratios were used to derive the odds ratio per 1 SD increments in suPAR levels instrumented by rs4760. Mendelian randomization analyses were performed using the ‘TwoSampleMR’ package in R version 4.0.

To assess whether rare coding variation with damaging consequences on the suPAR protein are associated with ischemic heart disease, in-silico lookup in exome-sequenced analysis of >280,000 UK Biobank participants was performed (Ref. 99; incorporated by reference in its entirety).

### SuPAR Over-Expression in a PCSK9-AAV Murine Model of Atherosclerosis

Male and female C57BL/6J wild-type mice (n=18) and suPAR^{Tg} mice (n=21) overexpressing the soluble form of mouse full-length suPAR (corresponding to NP_035243, DI-DII-DIII without GPI anchor) in adipose tissue using the adipocyte fatty acid binding protein (AP2 promoter) on C57BL/6 background (Ref. 10; incorporated by reference in its entirety). All mice were maintained on a 12-hour light-dark cycle with free access to food and water. To induce hypercholesterolemia, an intraperitoneal injection of recombinant adeno-associated virus 8-D377Y-murine Pcsk9 (5×10⁶ viral genomes/kg body weight) was administered (Ref. 100; incorporated by reference in its entirety). After 1 week, the diet was switched to a western diet (42% calories from fat, Teklad, catalog #88137) for 10-weeks. Animal experiments were carried out in accordance with the University of Michigan Institutional Animal Care and Use Committee.

### Cholesterol and SuPAR Measurements

Plasma was collected via tail vein puncture in heparin coated tubes. Fasting cholesterol levels were measured by colorimetric assay (Cell Biolabs, catalog #STA-384). Plasma levels of suPAR were measured using the R&D DuoSet ELISA antibodies and Ancillary Reagent kit 2 for development of a sandwich ELISA (R&D Systems, Minneapolis, MN, cat #DY531.) The ELISA has a detection range of 78-5000 μg/mL.

### Atherosclerotic Lesion Analysis, Histology, and Immune Histochemistry

Mice were euthanized via carbon dioxide overdose. Blood was harvested by right ventricular puncture and the vasculature perfused with ice-cold PBS. The heart and brachiocephalic artery (BCA) were harvested and placed in 4% paraformaldehyde and embedded in paraffin. Sixty sections (6 μm each) were cut through the aortic root as the primary site of atherosclerosis and 30 sections (6 μm each) were cut through the BCA as a secondary anatomic site as recommended from each mouse (Ref. 101; incorporated by reference in its entirety). For morphometric analysis, 30 sections from the aortic root and 15 sections from the BCA were stained with H&E and assessed for total lesion size and necrotic core size (acellular lesion area) as previously described (Ref. 102; incorporated by reference in its entirety), for a total coverage of 360 μm of the aortic root. Paraffin-embedded sections of the aortic sinus were deparaffinized and rehydrated.

After blocking, sections (6 μm each) were incubated at room temperature for 2 h with Mac2 (Santa Cruz Biotechnology, catalog #sc-81728; 1:100). Mac2 slides were counterstained with hematoxylin and cover-slipped. Images were captured with an Olympus LC30 camera mounted on Olympus CX41 microscope. For Mac2⁺ area, all images were obtained with the same light source at the same time. Mac2⁺ area was determined using the threshold function in ImageJ and normalized to total non-necrotic lesion area and reporting results as percentage of lesion area. Sectioning and staining were performed by the In Vivo Animal Core laboratory technicians within the Unit for Laboratory Animal Medicine at the University of Michigan. Technicians in this laboratory are blinded to the experimental identity. Atherosclerotic plaque size was calculated using ImageJ software (NIH, USA) and graphed by section number.

### Statistical Analysis for Murine Studies

All results are presented as mean±standard error of the mean (SEM). Comparisons in atherosclerotic lesion, necrotic core size, and total cholesterol level were made with 2-way ANOVA with Tukey's multiple comparison test (main effects). GraphPad Prism was used to perform statistical analysis and to generate figures.

### Results

SuPAR Levels Correlate with CAC and Predicts Increase in CAC
Study Cohort Characteristics Overall, MESA participants had a mean (SD) age of 63 (10) years), were 38.0% white and 48.5% male. The median (IQR) baseline suPAR levels were 2.5 (2.0, 3.1) ng/mL. With increasing suPAR categories, participants were older, more likely to be woman and have a history of smoking, diabetes and hypertension (P<0.001 for all) (Table 2). The median CAC for the cohort was 1.91 HU, and a total of 2636 (48.8%) participants had a CAC>0.

TABLE 2
Baseline characteristics of MESA cohort by suPAR categories

| | | | 0-2.0 | | 2.0-2.5 | | 2.5-3.0 | | >3.0 | | |
|---|---|---|---|---|---|---|---|---|---|---|---|
| | Total | | ng/mL | | ng/mL | | ng/mL | | ng/mL | | P- |
| | (n = 5,406) | | (n = 1456) | | (n = 1357) | | (n = 1099) | | (n = 1494) | | value |
| Demographics | | | | | | | | | | | |
| Age (years), mean | 63 | (10) | 59 | (9) | 62 | (10) | 65 | (10) | 65 | (11) | <0.001 |
| (SD) | | | | | | | | | | | |
| Race/ethnicity, n | | | | | | | | | | | <0.001 |
| (%) | | | | | | | | | | | |
| White | 2054 | (38.0) | 509 | (35.0) | 535 | (39.4) | 447 | (40.7) | 563 | (37.7) | |
| African American | 1464 | (27.1) | 421 | (28.9) | 328 | (24.2) | 270 | (24.6) | 445 | (29.8) | |
| Hispanic | 1221 | (22.6) | 205 | (14.1) | 341 | (25.1) | 293 | (26.7) | 382 | (25.6) | |
| Chinese American | 667 | (12.3) | 321 | (22.0) | 153 | (11.3) | 89 | (8.1) | 104 | (7.0) | |
| Male, n (%) | 2620 | (48.5) | 858 | (58.9) | 644 | (47.5) | 502 | (45.7) | 616 | (41.2) | <0.001 |
| Cardiac risk | | | | | | | | | | | |
| factors | | | | | | | | | | | |
| Body mass index, | 28 | (5) | 27 | (5) | 28 | (5) | 29 | (6) | 29 | (6) | <0.001 |
| mean (SD) | | | | | | | | | | | |
| Ever smoker, n (%) | 2664 | (49.5) | 656 | (45.3) | 651 | (48.1) | 560 | (51.2) | 797 | (53.5) | <0.001 |
| Diabetes, n (%) | 685 | (12.7) | 121 | (8.3) | 149 | (11.0) | 135 | (12.3) | 280 | (18.7) | <0.001 |
| Hypertension, n | 2448 | (45.3) | 536 | (36.8) | 563 | (41.5) | 499 | (45.4) | 850 | (56.9) | <0.001 |
| (%) | | | | | | | | | | | |
| Laboratory | | | | | | | | | | | |
| values, median | | | | | | | | | | | |
| (IQR) | | | | | | | | | | | |
| eGFR, mL/min | 77.3 | (66.2, 88.2) | 82.4 | (71.8, 92.6) | 78.3 | (67.9, 88.2) | 74.9 | (64.4, 86.2) | 72.0 | (60.4, 85.7) | <0.001 |
| Total cholesterol, | 192 | (170, 216) | 193 | (173, 215) | 195 | (172, 217) | 192 | (171, 216) | 188 | (165, 213) | <0.001 |
| mg/dL | | | | | | | | | | | |
| High-density | 48 | (40, 59) | 49 | (41, 60) | 50 | (41, 60) | 48 | (40, 59) | 46 | (39, 56) | <0.001 |
| cholesterol, mg/dL | | | | | | | | | | | |
| Low-density | 116 | (96, 136) | 118 | (98, 136) | 117 | (97, 136) | 116 | (95, 135) | 112 | (92, 136) | <0.001 |
| cholesterol, mg/dL | | | | | | | | | | | |
| suPAR, ng/mL | 2.5 | (2.0, 3.1) | 1.7 | (1.5, 1.9) | 2.3 | (2.1, 2.4) | 2.7 | (2.6, 2.9) | 3.7 | (3.3, 4.3) | <0.001 |
| Coronary artery | 1.9 | (0, 97.9) | 0 | (0, 47.2) | 0 | (0, 82.6) | 5.5 | (0, 148.0) | 12.2 | (0, 146.4) | <0.001 |
| calcium, HU | | | | | | | | | | | |

### Association Between Baseline CAC Scores and SuPAR

Participants with CAC>0 had significantly higher suPAR levels than those with CAC=0 (2.56 ng/mL, interquartile range IQR[2.05-3.23], compared to 2.34 ng/mL, IQR[1.89-2.95] respectively, P<0.001). Baseline suPAR levels correlated modestly with baseline CAC (Spearman rank r=0.14, P<0.001). After adjustment for demographics, cardiac risk factors, and laboratory data, higher baseline suPAR levels were significantly associated with a higher CAC score at baseline: for a 2-fold difference in suPAR levels, baseline CAC scores were higher by 27.3 HU (95% CI[6.92-47.8]) (Table 3).

TABLE 3
Association between baseline CAC score and suPAR levels

| | β^{a} (95% CI) | P-value |
|---|---|---|
| Age, years | 10.3 (9.1, 11.6) | <0.001 |
| Race/ethnicity, n (%) | | |
| White | 1.0 [reference] | |
| African American | −98.6 (−126.7, −70.5) | <0.001 |
| Hispanic | −69.8 (−99.2, −40.5) | <0.001 |
| Chinese American | −86.3 (−123.5, −49.2) | <0.001 |
| Male | 158.6 (134.3, 182.9) | <0.001 |
| Body mass index, kg/m² | 1.9 (−0.3, 4.2) | 0.08 |
| Ever smoker | 21.7 (−0.5, 44.0) | 0.06 |
| Diabetes | 87.3 (53.3, 121.3) | <0.001 |
| Hypertension | 63.5 (39.8, 87.2) | <0.001 |
| eGFR, per 5 mL/min | 5.4 (−2.5, 13.3) | 0.18 |
| High-density cholesterol, per 5 mg/dL | 2.8 (−5.6, 11.1) | 0.52 |
| Low-density cholesterol, per 10 mg/dL | 1.2 (−2.2, 4.6) | 0.50 |
| SuPAR | 27.3 (6.92, 47.8) | 0.009 |

^{a}Beta corresponds to each 2-fold difference in suPAR levels

Baseline suPAR and Longitudinal CAC

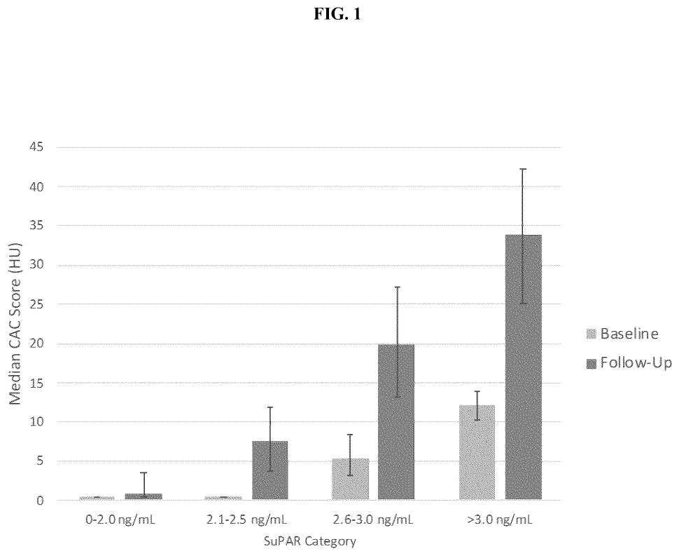

The median time between baseline and initial follow-up of CAC was 2.5 years. For all suPAR categories, the median CAC at follow-up was higher than baseline: participants with suPAR<2.0 ng/ml had a 103% increase in CAC at follow-up, compared to 229% for those with suPAR>3.0 ng/ml (FIG. 1). In multivariable analysis, higher baseline suPAR levels were associated with a greater increase in CAC scores over time (Table 4), with a yearly increase in CAC score of 15.1 HU (95% CI[6.7-23.5]) for a 100% increase in suPAR levels.

TABLE 4
Longitudinal association between baseline
suPAR levels and CAC score during follow-up

| | β^{a} (95% CI) | P-value |
|---|---|---|
| Age, years | 0.9 (0.4, 1.5) | <0.001 |
| Race/ethnicity, n (%) | | |
| White | 1.0 [reference] | |
| African American | −5.9 (−18.3, 6.5) | 0.35 |
| Hispanic | −9.1 (−22.3, 4.1) | 0.18 |
| Chinese American | −12.2 (−24.4, −0.0) | 0.049 |
| Male | 13.5 (4.2, 22.9) | 0.005 |
| Body mass index, kg/m² | 0.7 (−0.3, 1.8) | 0.18 |
| Ever smoker | 4.0 (−4.6, 12.6) | 0.37 |
| Diabetes | 19.3 (11.9, 26.6) | <0.001 |
| Hypertension | 19.3 (10.3, 28.4) | <0.001 |
| eGFR, per 5 mL/min | −1.6 (−5.7, 2.5) | 0.45 |
| High-density cholesterol, per 5 mg/dL | −1.5 (−4.8, 1.8) | 0.37 |
| Low-density cholesterol, per 10 mg/dL | 0.01 (−1.4, 1.4) | 0.98 |
| Baseline CAC | 1.3 (1.2, 1.3) | <0.001 |
| Follow-up, years | 15.4 (5.0, 25.7) | 0.004 |
| SuPAR | −23.9 (−37.6, −10.3) | <0.001 |
| SuPAR*follow-up | 15.1 (6.7, 23.5) | <0.001 |

^{a}Beta corresponds to each 2-fold difference in suPAR levels

### SuPAR and Cardiovascular Outcomes

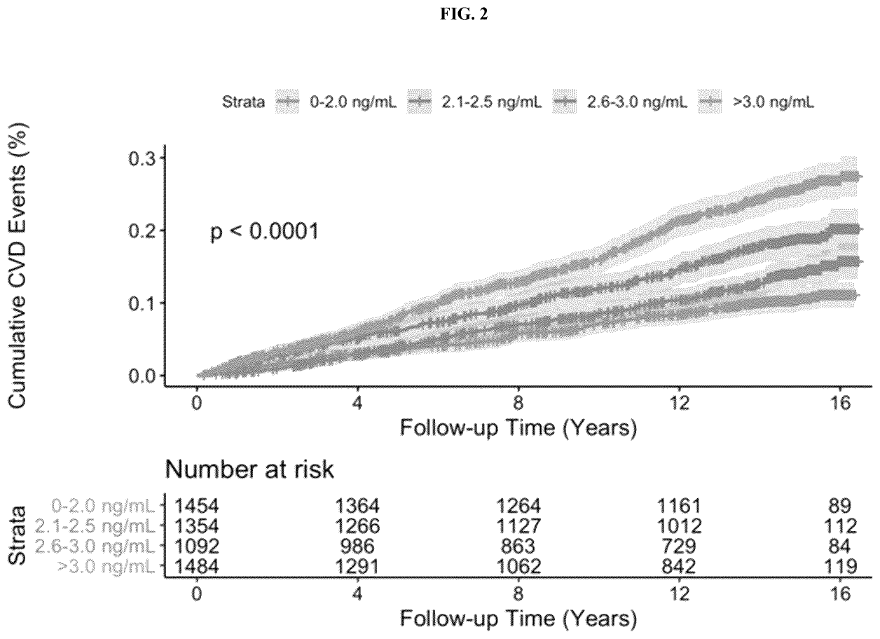

A total of 604 (11.2%) participants developed incident CVD over a median follow-up time of 15 years (8.9 events per 1,000 person-years). Higher suPAR categories were associated with a higher incidence of CVD events (log-rank P<0.001) (FIG. 2). Participants with a baseline suPAR >3.0 ng/mL had an incidence rate of 9.0 CVD events per 1,000 person-years (95% CI[7.7-10.7]), whereas participants with a baseline suPAR between 0-2.0 ng/mL had an incidence rate of 2.8 events per 1,000 person-years (95% CI [2.2-3.7]). In multivariable analysis, higher suPAR levels were associated with a higher risk of CVD events: 1.49-fold higher (95% CI[1.32-1.68]) for each 2-fold higher suPAR level, and 1.83-fold higher (95% CI[1.58-2.27]) for participants with suPAR>3.0 ng/mL compared to suPAR<2.0 ng/mL (Table 5). The association between suPAR and CVD events was not attenuated by adjusting for eGFR or baseline CAC (FIG. 6), and did not differ according to the presence (CAC>0) or absence of CAC (CAC=0) at baseline (P interaction=0.31) or baseline eGFR (P interaction=0.98).

TABLE 5
Survival analysis for baseline suPAR levels and CVD events

| | | HR (95% CI) | P-value |
|---|---|---|---|
| Age, years | | 1.06 (1.05, 1.07) | <0.001 |
| Race/ethnicity, n (%) | | | |
| White | | 1.0 [reference] | |
| African American | | 0.83 (0.69, 1.00) | 0.045 |
| Hispanic | | 0.98 (0.81, 1.17) | 0.79 |
| Chinese American | | 0.85 (0.66, 1.09) | 0.19 |
| Male | | 1.76 (1.50, 2.06) | <0.001 |
| Body mass index, per 5 kg/m² | | 1.03 (0.95, 1.11) | 0.45 |
| Ever smoker | | 1.14 (0.98, 1.31) | 0.08 |
| Diabetes | | 1.69 (1.42, 2.02) | <0.001 |
| Hypertension | | 1.83 (1.56, 2.14) | <0.001 |
| eGFR, per 5 mL/min | | 1.01 (0.96, 1.06) | 0.74 |
| High-density cholesterol, per 5 mg/dL | | 0.94 (0.88, 0.99) | 0.029 |
| Low-density cholesterol, per 10 mg/dL | | 1.04 (1.01, 1.06) | 0.002 |
| SuPAR, log-base 2 | | 1.49 (1.32, 1.68) | <0.001 |
| SuPAR, categorical | | | <0.001 |
| 0-2.0 | ng/mL | 1.0 [reference] | |
| 2.0-2.5 | ng/mL | 1.14 (0.90, 1.43) | 0.27 |
| 2.5-3.0 | ng/mL | 1.41 (1.12, 1.77) | 0.003 |
| >3.0 | ng/mL | 1.83 (1.58, 2.27) | <0.001 |

Number of cardiovascular disease (CVD) events was 594. A CVD event was defined as the composite of myocardial infarction, resuscitated cardiac arrest, angina, revascularization, stroke (excluding transient ischemic attack), or death due to CVD.

Genome-Wide Association Meta-Analysis of suPAR

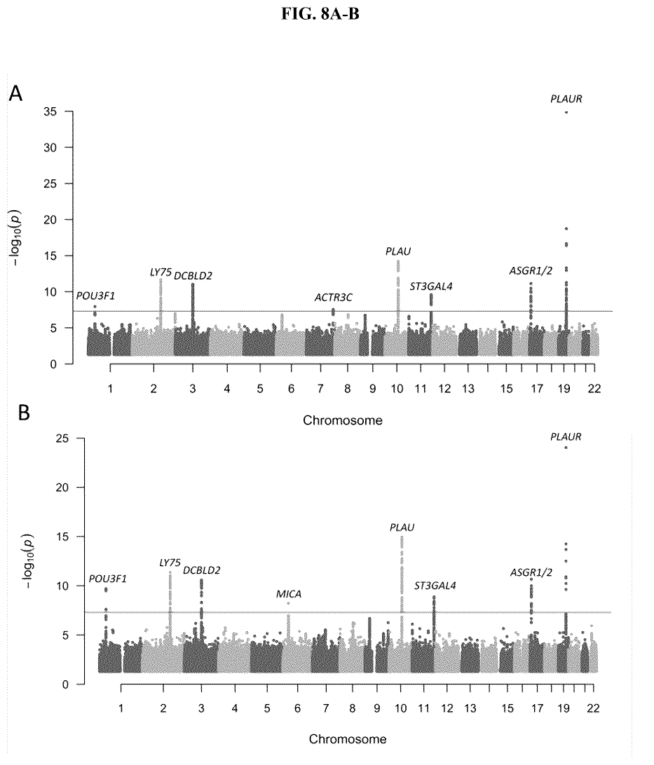

A multi-ancestry GWAS meta-analysis of suPAR levels was performed (FIG. 7) on 16.6 million variants in 12,937 individuals from European (n=9,869), African (n=1,363), East Asian (n=623) and Hispanic (n=1,082) ancestries. Fifteen independent signals in 8 loci were associated with suPAR levels at a genome-wide significance level (P<5×10⁻⁸) (Table 1, FIG. 8). A meta-analysis limited to the European ancestry sample included 9.9 million variants and identified 12 independent signals in 8 loci at genome-wide significance (FIG. 8, Table 5).

TABLE 1
Top variants from multi-ancestry genome-wide association analysis of suPAR

| | | | | | | | | | | DBDS | | |
|---|---|---|---|---|---|---|---|---|---|---|---|---|
| | | | | | | | TSS, GABC, MESA, MDC | | | (n = 12,177) | | |
| | | | | | | | (n = 12,937) | | | | | P- |
| SNP | Chromosome | Position | Locus | EA | OA | EAF | Effect | SE | P-value | Effect | SE | value |
| rs60104061 | 1 | 38093161 | POU3F1 | G | A | 0.03 | −0.21 | 0.04 | 1 × 10⁻⁸ | 0.03 | 0.04 | 0.45 |
| rs925411 | 2 | 159889274 | LY75 | T | G | 0.40 | −0.09 | 0.01 | 2 × 10⁻¹² | −0.10 | 0.01 | 6 × 10⁻¹⁶ |
| rs9821965 | 3 | 98979570 | DCBLD2 | G | A | 0.36 | −0.08 | 0.01 | 7 × 10⁻¹¹ | −0.10 | 0.01 | 7 × 10⁻¹⁶ |
| rs9836915 | 3 | 98906459 | DCBLD2 | T | C | 0.04 | −0.22 | 0.03 | 9 × 10⁻¹² | −0.20 | 0.03 | 7 × 10⁻¹⁰ |
| rs11982709 | 7 | 150225754 | ACTR3C | A | G | 0.01 | −0.59 | 0.11 | 3 × 10⁻⁰⁸ | −0.16 | 0.21 | 0.45 |
| rs2633321 | 10 | 73933937 | PLAU | A | G | 0.50 | 0.10 | 0.01 | 6 × 10⁻¹⁵ | 0.11 | 0.01 | 3 × 10⁻¹⁹ |
| rs9704688 | 11 | 126372477 | ST3GAL4 | T | C | 0.18 | −0.11 | 0.02 | 3 × 10⁻¹⁰ | −0.14 | 0.02 | 1 × 10⁻¹³ |
| rs535064984 | 17 | 7116978 | ASGR2 | C | T | 0.004 | 0.93 | 0.17 | 4 × 10⁻⁸ | 0.54 | 0.09 | 4 × 10⁻⁹ |
| rs55714927 | 17 | 7176997 | ASGR1 | T | C | 0.16 | 0.11 | 0.02 | 7 × 10⁻¹² | 0.12 | 0.02 | 2 × 10⁻¹⁴ |
| rs117564136 | 19 | 43673548 | PLAUR | T | C | 0.06 | 0.13 | 0.02 | 4 × 10⁻⁸ | 0.05 | 0.02 | 0.03 |
| rs3213247 | 19 | 43574584 | PLAUR | A | C | 0.04 | 0.17 | 0.03 | 6 × 10⁻⁹ | 0.04 | 0.03 | 0.11 |
| rs36229204 | 19 | 43671830 | PLAUR | T | C | 0.03 | −0.23 | 0.03 | 1 × 10⁻¹¹ | −0.24 | 0.03 | 2 × 10⁻¹⁷ |
| rs3760977 | 19 | 43670267 | PLAUR | T | C | 0.003 | 1.21 | 0.14 | 2 × 10⁻¹⁷ | — | — | — |
| rs2302524 | 19 | 43652320 | PLAUR | C | T | 0.17 | 0.21 | 0.02 | 1 × 10⁻³⁵ | 0.10 | 0.02 | 3 × 10⁻⁸ |
| rs4760 | 19 | 43648948 | PLAUR | G | A | 0.10 | 0.11 | 0.02 | 8 × 10⁻⁹ | 0.08 | 0.02 | 6 × 10⁻⁶ |

Abbreviations: GABC: Genes of Blood-Clotting Cohort; DBDS: Danish Blood Donor Study; EA, Effect Allele; EAF, Effect Allele Frequency; MESA: Multi-Ethnic Study of Atherosclerosis; MDC: Malmo Diet and Cancer study; OA, Other Allele; SE, Standard Error; SNP, Single Nucleotide Polymorphism; TSS: Trinity Student Study.

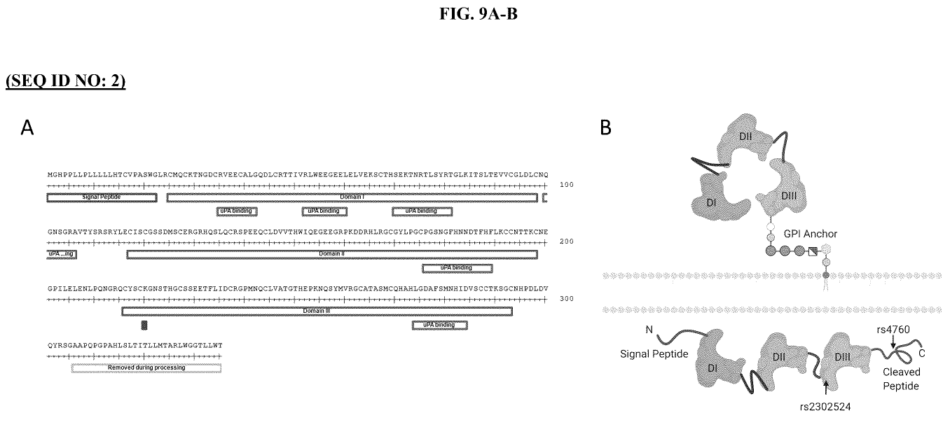

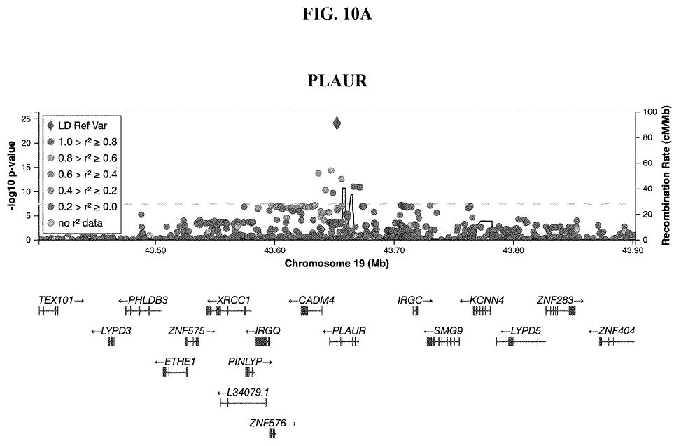

Included in the 8 significantly associated loci were variants in or near the genes encoding suPAR (PLAUR) and its canonical ligand uPA (PLAU). Two missense variants in the PLAUR gene were identified (FIG. 9): rs2302524 (p.Lys220Arg in domain III of uPAR); with each minor C allele associated with 0.21 SD incremental increase in suPAR levels (P=1×10⁻³⁵), and rs4760 (p.Leu317Pro in the c-terminal portion of suPAR) with each minor G allele associated with 0.11 SD increase in suPAR levels (P=8×10⁻⁹). Four other putatively independent signals were tagged by top SNPs in the non-coding sequence of the PLAUR locus (Table 1). At the PLAU locus, the A allele of rs2633321 was associated with higher levels of suPAR (0=0.10 SD, P=6×10⁻⁵). Associations between suPAR levels and 12 of the 15 signals, including the two PLAUR missense variants, were replicated in the DBDS cohort (Table 1, Table 6).

TABLE 6
Top variants from genome-wide association analysis of suPAR in participants with European ancestry.

| | | | | | | | TSS, GABC, MESA, MDC | | | DBDS | | |
|---|---|---|---|---|---|---|---|---|---|---|---|---|
| | | | | | | | (n = 12,937) | | | (n = 12,177) | | |
| | C- | | | | | | | | P- | | | P- |
| SNP | some | Position | Locus | EA | OA | EAF | Effect | SE | value | Effect | SE | value |
| rs12077698 | 1 | 38088049 | POU3F1 | C | G | 0.08 | −0.23 | 0.04 | 2 × 10⁻¹⁰ | 0.00 | 0.04 | 0.96 |
| rs7595388 | 2 | 159899834 | LY75 | A | G | 0.34 | −0.10 | 0.01 | 4 × 10⁻¹² | −0.10 | 0.01 | 5 × 10⁻¹⁴ |
| rs56172805 | 3 | 98907345 | DCBLD2 | A | G | 0.05 | −0.22 | 0.03 | 3 × 10⁻¹¹ | −0.20 | 0.03 | 7 × 10⁻¹⁰ |
| rs79493037 | 6 | 31438048 | MICA | G | C | 0.13 | −0.18 | 0.03 | 6 × 10⁻⁰⁹ | −0.06 | 0.03 | 0.02 |
| rs2633313 | 10 | 73924107 | PLAU | C | T | 0.46 | −0.12 | 0.01 | 1 × 10⁻¹⁵ | −0.11 | 0.01 | 1 × 10⁻¹⁸ |
| rs240559 | 11 | 126321217 | ST3GAL4 | T | C | 0.21 | 0.09 | 0.02 | 3 × 10⁻⁰⁸ | 0.05 | 0.01 | 0.001 |
| rs3967200 | 11 | 126362490 | ST3GAL4 | T | C | 0.17 | −0.13 | 0.02 | 1 × 10⁻⁰⁹ | −0.14 | 0.02 | 2 × 10⁻¹⁴ |
| rs535064984 | 17 | 7116978 | ASGR2 | C | T | 0.004 | −0.93 | 0.17 | 4 × 10⁻⁰⁸ | 0.54 | 0.09 | 4 × 10⁻⁰⁹ |
| rs55714927 | 17 | 7176997 | ASGR1 | T | C | 0.16 | 0.12 | 0.02 | 2 × 10⁻¹¹ | 0.12 | 0.02 | 2 × 10⁻¹⁴ |
| rs4251824 | 19 | 43666441 | PLAUR | T | C | 0.04 | −0.24 | 0.04 | 1 × 10⁻¹¹ | −0.24 | 0.03 | 4 × 10⁻¹⁷ |
| rs2302524 | 19 | 43652320 | PLAUR | C | T | 0.17 | 0.20 | 0.02 | 9 × 10⁻²⁵ | 0.10 | 0.02 | 3 × 10⁻⁸ |
| rs4760 | 19 | 43648948 | PLAUR | G | A | 0.10 | 0.13 | 0.02 | 2 × 10⁻¹⁰ | 0.08 | 0.02 | 6 × 10⁻⁶ |

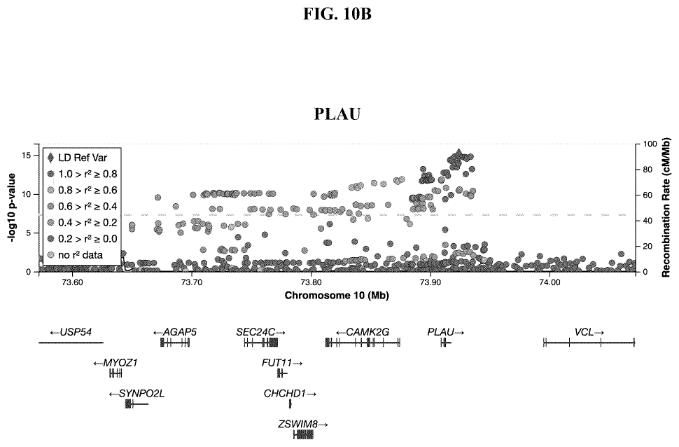

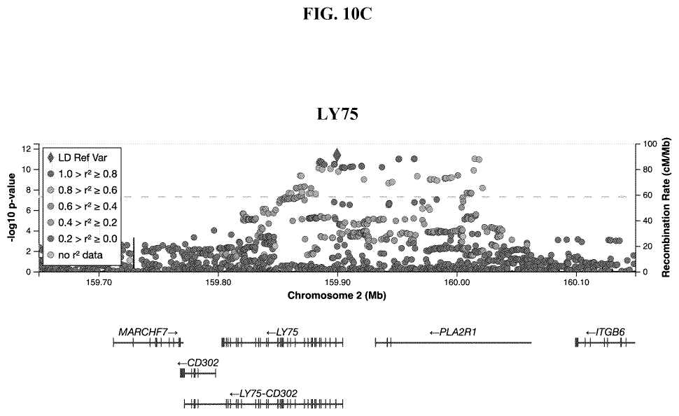

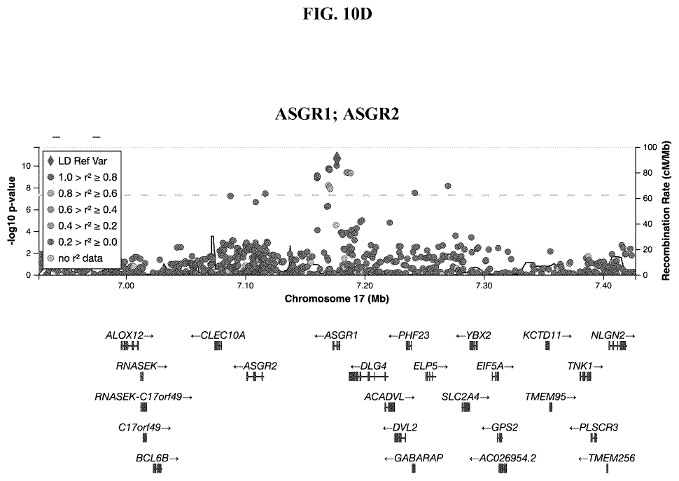

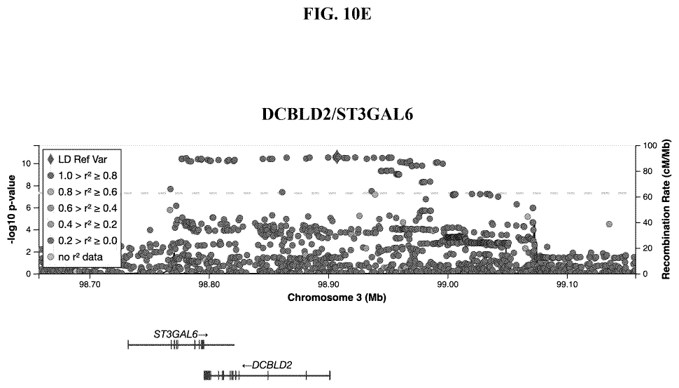

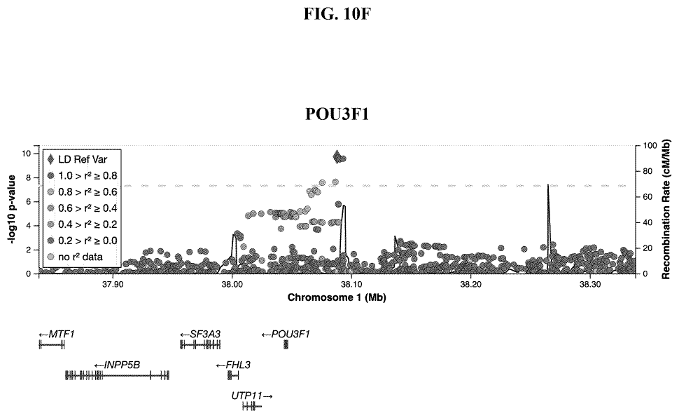

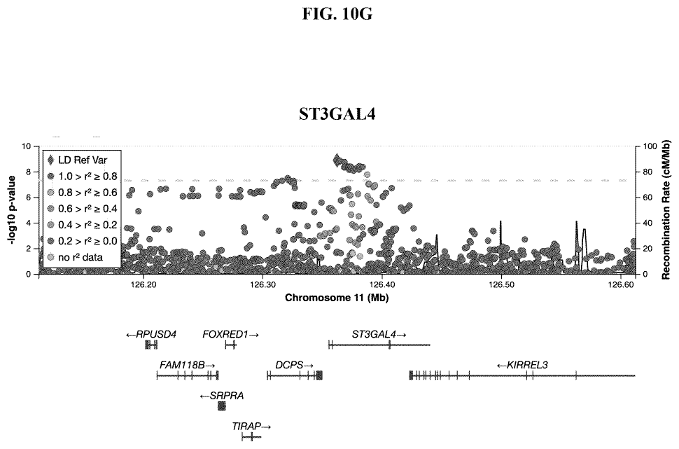

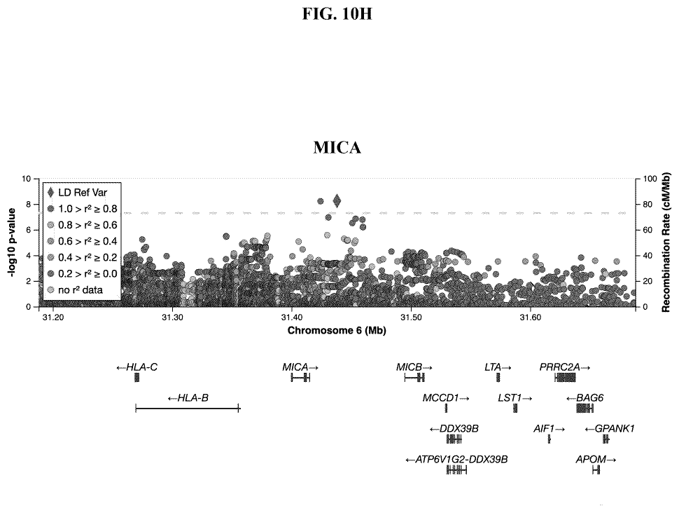

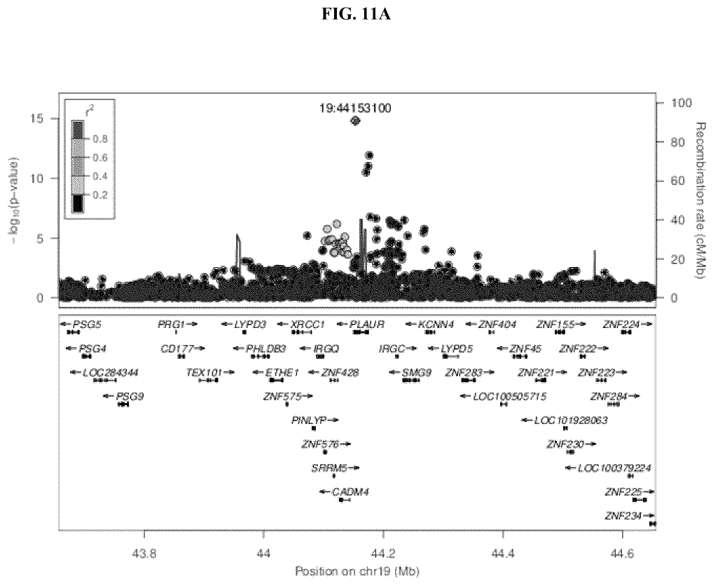

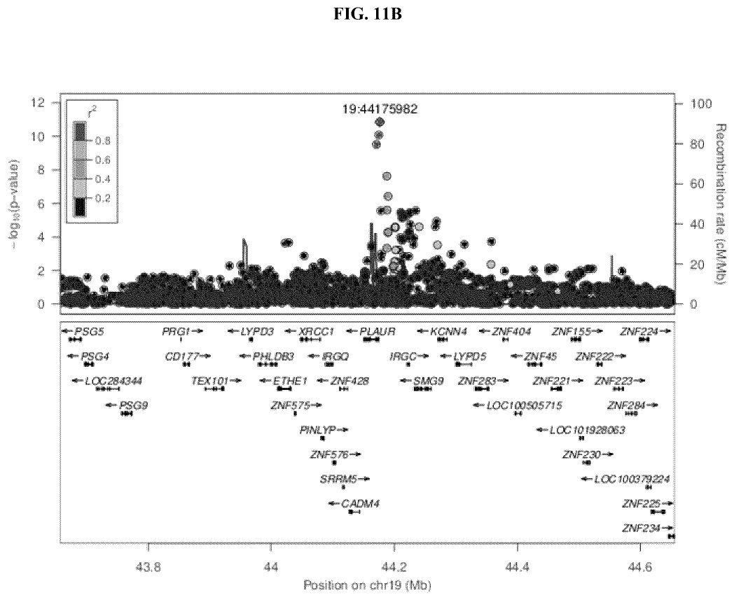

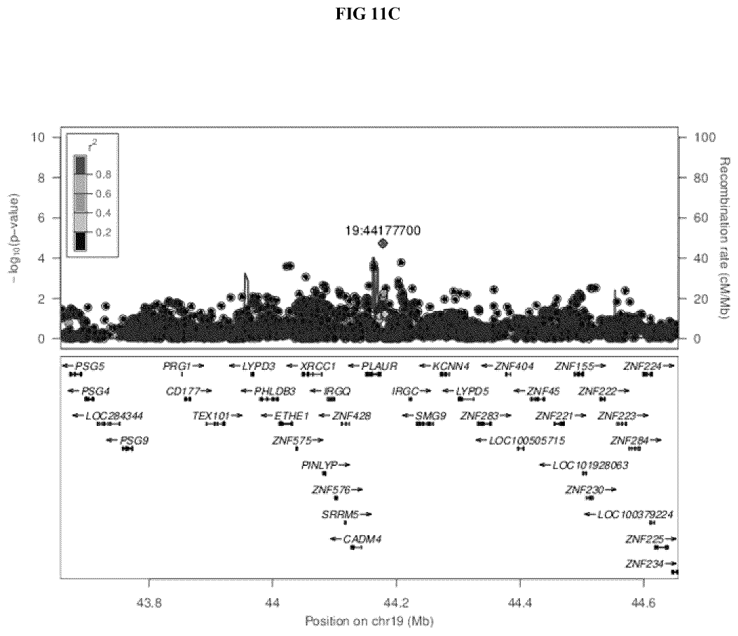

The regional association plots for all 8 loci from the European ancestry meta-analysis are illustrated in FIG. 10. The variance of suPAR levels explained by a weighted genetic risk score of all independent variants was 3%. Using sequential conditional analysis, 3 of the top 6 variants at the PLAUR locus (rs4760, rs2302524 and rs36229204) remained independent (FIG. 11).

Impact of PLAUR Missense Variants on suPAR Levels

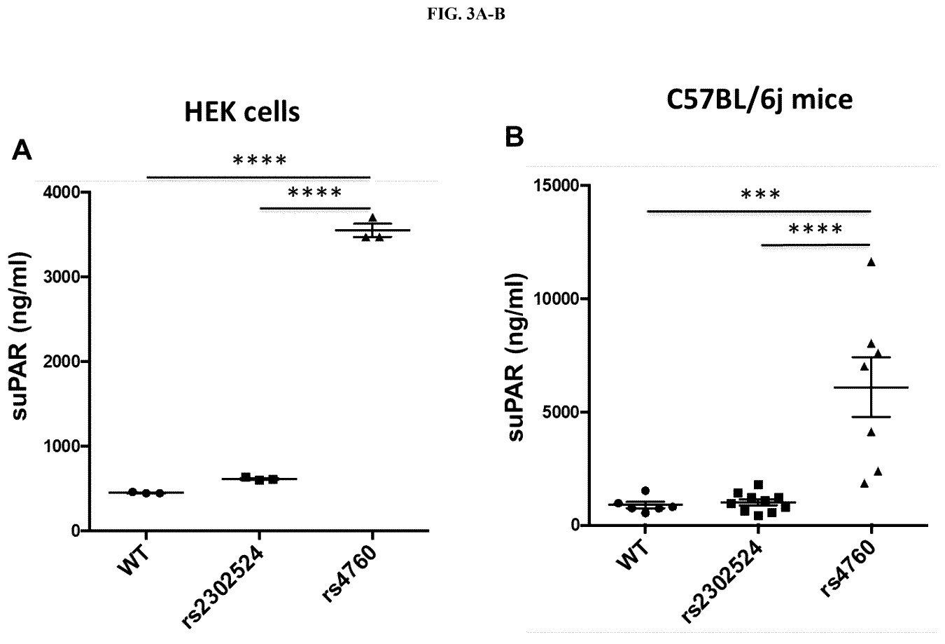

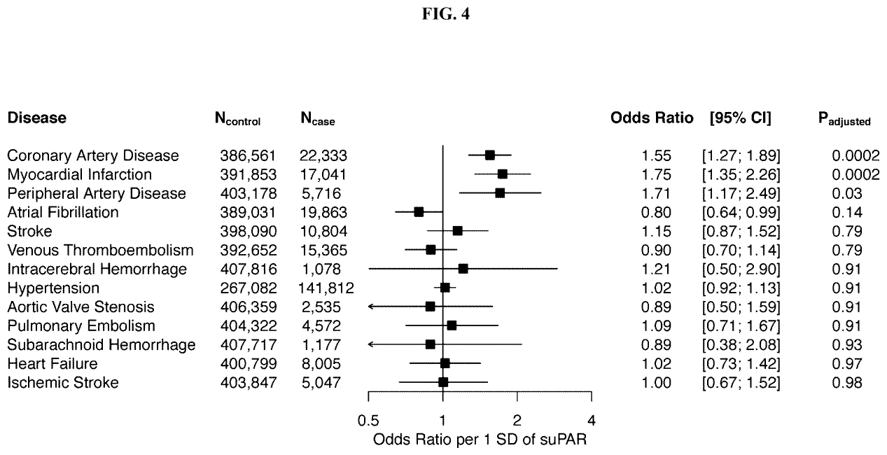

Given their likelihood of altering suPAR levels, it was assessed experimentally whether the PLAUR missense variants rs2302524 and rs4760 lead to altered suPAR levels compared to the reference allele. HEK293 cells were transfected with plasmid DNA encoding either reference cDNA or missense variants and measured suPAR levels in the supernatant 48-hours later. The supernatant of cells transfected with the rs4760 variant had 8-fold higher suPAR levels compared with reference; while no increase in suPAR was observed in the medium of cells transfected with rs2302524 (FIG. 3). Expression of rs4760 (p.317Pro) in-vivo using mouse hydrodynamic tail vein injection of the plasmid DNA similarly showed a close to 7-fold increase in serum suPAR levels 24 hours after injection, while rs2302524 had no significant impact (FIG. 3).

Genetically Predicted suPAR Level and Atherosclerotic Disease

To assess whether suPAR levels are causally linked to atherosclerotic disease, MR was performed using the PLAUR rs4760 missense variant and the following phenotypes: aortic valve stenosis, atrial fibrillation, coronary artery disease, heart failure, hypertension, intracerebral hemorrhage, ischemic stroke, myocardial infarction, peripheral artery disease, pulmonary embolism, stroke, subarachnoid hemorrhage, venous thromboembolism. Genetically predicted 1-SD increment in suPAR was associated with 55% higher odds of coronary artery disease (P_{adjusted}=0.0002), 75% higher odds of myocardial infarction (P_{adjusted}=0.0002) and 71% peripheral vascular disease (P_{adjusted}=0.03) after adjusting for multiple comparisons (FIG. 4).

Rare variant gene collapsing analysis of the >280,000 exomes in the UK Biobank indicated that individuals with rare non-benign missense variants in PLAUR (minor allele frequency less than 0.0005)—had a lower risk of ischemic heart disease (OR 0.59, 95% CI[0.37-0.93]), indicating that haploinsufficiency of suPAR is protective against ischemic heart disease.

### SuPAR Over-Expression Exacerbates Atherosclerosis

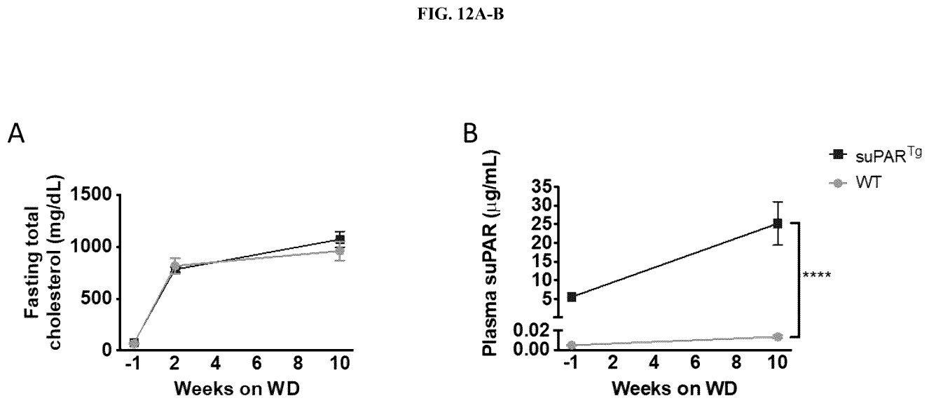

Male and female hypercholesterolemic suPAR overexpressing mice (suPAR^{Tg}) had similar total cholesterol levels at baseline and after D377Y-m Pcsk9 overexpression coupled with western diet feeding for 10 weeks compared to wild-type (WT) mice (FIG. 12). SuPAR levels were significantly higher in suPAR^{Tg} mice compared to WT at baseline (2.4 μg/mL vs. 0.005 μg/mL, respectively) and at 10 weeks (25.2 μg/mL vs. 0.01 μg/mL, respectively; FIG. 12)

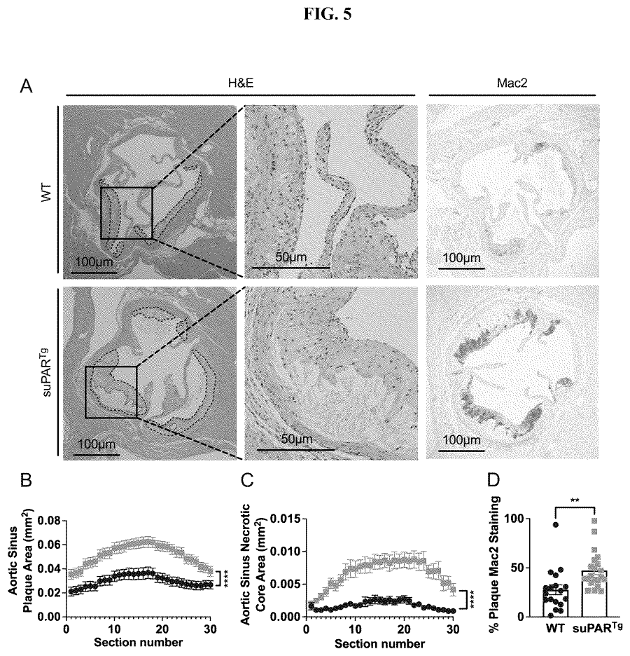

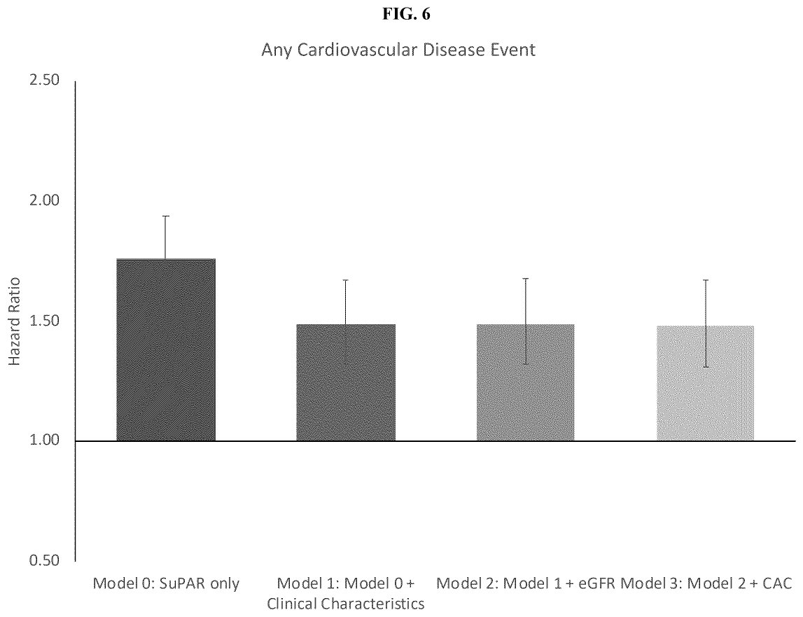

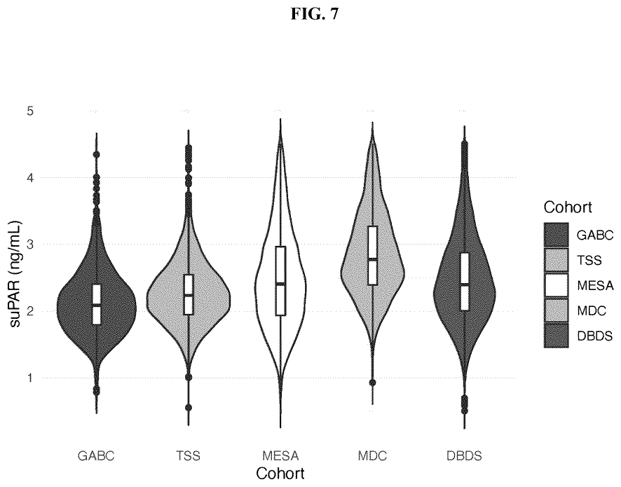

All suPAR^{Tg} (n=21) mice developed larger plaques in the aortic root compared to the WT group (n=18); with a mean plaque volume of 1.55 mm³ in the suPAR T and 0.90 mm³ in the WT group (FIG. 5). The necrotic area within the atherosclerotic plaques of the suPAR T mice had significantly increased necrotic core areas compared with WT mice with a mean volume of 0.20 mm³ compared to 0.052 mm³, respectively. Furthermore, the atherosclerotic plaques of the suPAR^{Tg} mice had a significantly higher percentage of macrophage positive area with by Mac2 staining of 47.3% versus 27.6% in the WT mice (FIG. 5).

Elevated suPAR Levels Induce Pro-Atherogenic Changes in Monocyte Profiles

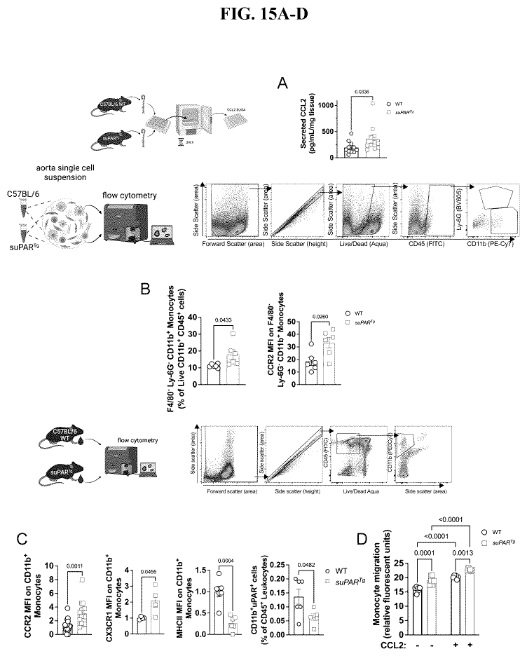

Given the role of the urokinase receptor system in the regulation of innate immune system physiology, notably efferocytosis, experiments were conducted to assess whether suPAR over-expression altered the profile and function of monocytes and macrophages. To that end, aortas were isolated from wild-type and suPAR^{Tg} mice who did not undergo PCSK9-AAV transfection, as to avoid the confounding effects of atherosclerosis and hyperlipidemia, were examined. It was found that non-atherosclerotic suPAR^{Tg} aortas secreted significantly higher levels of C-C Motif Chemokine Ligand 2 (CCL2)—one of the primary monocyte chemo-attractants implicated in atherosclerosis—compared to wild-type aortas (FIG. 15A). Flow cytometry of aortic cell suspensions revealed a 2-fold higher count of monocytes in suPAR^{Tg} aortas compared to wild-type (FIG. 15B). The suPAR^{Tg} monocytes isolated from aortas exhibited higher expression of C-C chemokine receptor type 2 (CCR2)—the receptor for CCL2—compared to wild-type monocytes (FIG. 15B). Circulating monocytes and bone-marrow derived macrophages exhibited a similarly pro-inflammatory phenotype with higher expression of CCR2 and lower expression of major histocompatibility complex class 2 (MHCII) and membrane bound uPAR (FIG. 15C,). Circulating monocytes from suPAR^{Tg} mice also exhibited increased expression of Cx3CR1, another chemokine receptor that has been implicated in atherosclerosis, compared to wild-type (FIG. 15C).

It was assessed whether monocyte chemotaxis in suPAR^{Tg} is altered as measured by migratory potential using a trans-well assay. Significantly more suPAR^{Tg} monocytes migrated through the trans-well membrane compared to wild-type monocytes in response to both basal cell culture media as well as cell culture media with added recombinant CCL2 (FIG. 15D). Overall, these data indicate that suPAR acts on monocytes, and myeloid cells in general, to render these cells more atherogenic.

### REFERENCES

The following references, some of which have been cited above by number, are incorporated by reference in their entireties.
1. Saran, R., et al. US Renal Data System 2018 Annual Data Report: Epidemiology of Kidney Disease in the United States. Am J Kidney Dis 73, A7-A8 (2019).
2. Gansevoort, R. T., et al. Chronic kidney disease and cardiovascular risk: epidemiology, mechanisms, and prevention. Lancet 382, 339-352 (2013).
3. Solberg, H., Ploug, M., Hoyer-Hansen, G., Nielsen, B. S. & Lund, L. R. The murine receptor for urokinase-type plasminogen activator is primarily expressed in tissues actively undergoing remodeling. J Histochem Cytochem 49, 237-246 (2001).
4. Plesner, T., et al. Expression of the receptor for urokinase-type plasminogen activator in normal and neoplastic blood cells and hematopoietic tissue. Am J Clin Pathol 102, 835-841 (1994).
5. Nykjaer, A., et al. Urokinase receptor. An activation antigen in human T lymphocytes. J Immunol 152, 505-516 (1994).
6. Isselbacher, E. M. Thoracic and abdominal aortic aneurysms. Circulation 111, 816-828 (2005).
7. Thuno, M., Macho, B. & Eugen-Olsen, J. suPAR: the molecular crystal ball. Dis Markers 27, 157-172 (2009).
8. Wei, C., et al. Modification of kidney barrier function by the urokinase receptor. Nat Med 14, 55-63 (2008).
9. Huai, Q., et al. Structure of human urokinase plasminogen activator in complex with its receptor. Science 311, 656-659 (2006).
10. Hahm, E., et al. Bone marrow-derived immature myeloid cells are a main source of circulating suPAR contributing to proteinuric kidney disease. Nat Med 23, 100-106 (2017).
11. Hayek, S. S., et al. Cardiovascular Disease Biomarkers and suPAR in Predicting Decline in Renal Function: A Prospective Cohort Study. Kidney Int Rep 2, 425-432 (2017).
12. Hayek, S. S., et al. A tripartite complex of suPAR, APOL1 risk variants and alphavbeta3 integrin on podocytes mediates chronic kidney disease. Nat Med 23, 945-953 (2017).
13. Hayek, S. S., et al. Soluble Urokinase Plasminogen Activator Receptor and Decline in Kidney Function in Autosomal Dominant Polycystic Kidney Disease. J Am Soc Nephrol 30, 1305-1313 (2019).
14. Hayek, S. S., et al. Soluble Urokinase Receptor and Chronic Kidney Disease. N Engl J Med 373, 1916-1925 (2015).
15. Luo, S., et al. Soluble Urokinase-Type Plasminogen Activator Receptor in Black Americans with CKD. Clin J Am Soc Nephrol 13, 1013-1021 (2018).
16. Schaefer, F., et al. Association of Serum Soluble Urokinase Receptor Levels With Progression of Kidney Disease in Children. JAMA Pediatr 171, e172914 (2017).
17. Wei, C., et al. uPAR isoform 2 forms a dimer and induces severe kidney disease in mice. J Clin Invest (2019).
18. Hayek, S. S., et al. Soluble Urokinase Receptor and Acute Kidney Injury. N Engl J Med 382, 416-426 (2020).
19. Schulz, C. A., et al. Soluble Urokinase-type Plasminogen Activator Receptor (suPAR) and Impaired Kidney Function in the Population-based Malmo Diet and Cancer Study. Kidney Int Rep 2, 239-247 (2017).
20. Wei, C., et al. Circulating urokinase receptor as a cause of focal segmental glomerulosclerosis. Nat Med 17, 952-960 (2011).
21. Wei, C., et al. uPAR isoform 2 forms a dimer and induces severe kidney disease in mice. J Clin Invest 129, 1946-1959 (2019).
22. Ramos, M., Lao, Y., Eguiluz, C., Del Val, M. & Martinez, I. Urokinase receptor-deficient mice mount an innate immune response to and clarify respiratory viruses as efficiently as wild-type mice. Virulence 6, 710-715 (2015).
23. Spinale, J. M., et al. A reassessment of soluble urokinase-type plasminogen activator receptor in glomerular disease. Kidney Int 87, 564-574 (2015).
24. Dal Monte, M., et al. Inhibiting the urokinase-type plasminogen activator receptor system recovers STZ-induced diabetic nephropathy. J Cell Mol Med 23, 1034-1049 (2019).
25. Eugen-Olsen, J., Ladelund, S. & Sorensen, L. T. Plasma suPAR is lowered by smoking cessation: a randomized controlled study. Eur J Clin Invest 46, 305-311 (2016).
26. Haupt, T. H., et al. Healthy lifestyles reduce suPAR and mortality in a Danish general population study. Immun Ageing 16, 1 (2019).
27. Hayek, S. S., et al. Predicting Mortality in African Americans With Type 2 Diabetes Mellitus: Soluble Urokinase Plasminogen Activator Receptor, Coronary Artery Calcium, and High-Sensitivity C-Reactive Protein. J Am Heart Assoc 7(2018).
28. Edsfeldt, A., et al. Soluble urokinase plasminogen activator receptor is associated with inflammation in the vulnerable human atherosclerotic plaque. Stroke 43, 3305-3312 (2012).
29. Samman Tahhan, A., et al. Circulating soluble urokinase plasminogen activator receptor levels and peripheral arterial disease outcomes. Atherosclerosis 264, 108-114 (2017).
30. Botha, S., et al. Soluble urokinase plasminogen activator receptor as a prognostic marker of all-cause and cardiovascular mortality in a black population. Int J Cardiol 184, 631-636 (2015).
31. Cyrille, N. B., Villablanca, P. A. & Ramakrishna, H. Soluble urokinase plasminogen activation receptor—An emerging new biomarker of cardiovascular disease and critical illness. Ann Card Anaesth 19, 214-216 (2016).
32. Eapen, D. J., et al. Soluble urokinase plasminogen activator receptor level is an independent predictor of the presence and severity of coronary artery disease and of future adverse events. J Am Heart Assoc 3, e001118 (2014).
33. Eugen-Olsen, J., et al. Circulating soluble urokinase plasminogen activator receptor predicts cancer, cardiovascular disease, diabetes and mortality in the general population. J Intern Med 268, 296-308 (2010).
34. Haupt, T. H., et al. Plasma suPAR levels are associated with mortality, admission time, and Charlson Comorbidity Index in the acutely admitted medical patient: a prospective observational study. Crit Care 16, R130 (2012).
35. Lyngbaek, S., et al. Cardiovascular risk prediction in the general population with use of suPAR, CRP, and Framingham Risk Score. Int J Cardiol 167, 2904-2911 (2013).
36. Meijers, B., et al. Soluble urokinase receptor is a biomarker of cardiovascular disease in chronic kidney disease. Kidney Int 87, 210-216 (2015).
37. Donadello, K., et al. Soluble urokinase-type plasminogen activator receptor as a prognostic biomarker in critically ill patients. J Crit Care 29, 144-149 (2014).
38. Eapen, D. J., et al. Soluble urokinase plasminogen activator receptor level is an independent predictor of the presence and severity of coronary artery disease and of future adverse events. Journal of the American Heart Association 3, e001118 (2014).
39. Ghasemzedah, N., et al. Pathway-Specific Aggregate Biomarker Risk Score Is Associated With Burden of Coronary Artery Disease and Predicts Near-Term Risk of Myocardial Infarction and Death. Circ Cardiovasc Qual Outcomes 10(2017).
40. Drechsler, C., et al. Soluble Urokinase Plasminogen Activator Receptor and Outcomes in Patients with Diabetes on Hemodialysis. Clin J Am Soc Nephrol 12, 1265-1273 (2017).
41. Amdur, R. L., et al. Use of Measures of Inflammation and Kidney Function for Prediction of Atherosclerotic Vascular Disease Events and Death in Patients With CKD: Findings From the CRIC Study. Am J Kidney Dis 73, 344-353 (2019).
42. Zoccali, C., et al. The systemic nature of CKD. Nat Rev Nephrol 13, 344-358 (2017).
43. Ridker, P. M., et al. Inhibition of Interleukin-1beta by Canakinumab and Cardiovascular Outcomes in Patients With Chronic Kidney Disease. J Am Coll Cardiol 71, 2405-2414 (2018).
44. Hodges, G., et al. SuPAR is associated with death and adverse cardiovascular outcomes in patients with suspected coronary artery disease. Scand Cardiovasc J54, 339-345 (2020).
45. Sorensen, M. H., et al. Soluble urokinase plasminogen activator receptor is in contrast to high-sensitive C-reactive-protein associated with coronary artery calcifications in healthy middle-aged subjects. Atherosclerosis 237, 60-66 (2014).
46. Wu, W., et al. Soluble Urokinase Plasminogen Activator Receptor is Associated with Coronary Artery Calcification and Cardiovascular Disease in Patients Undergoing Hemodialysis. Kidney Blood Press Res 43, 664-672 (2018).
47. Diederichsen, M. Z., et al. Prognostic value of suPAR and hs-CRP on cardiovascular disease. Atherosclerosis 271, 245-251 (2018).
48. Hamirani, Y. S., et al. Markers of inflammation and coronary artery calcification: a systematic review. Atherosclerosis 201, 1-7 (2008).
49. Jenny, N. S., et al. Associations of inflammatory markers with coronary artery calcification: results from the Multi-Ethnic Study of Atherosclerosis. Atherosclerosis 209, 226-229 (2010).
50. Visscher, P. M., et al. 10 Years of GWAS Discovery: Biology, Function, and Translation. Am J Hum Genet 101, 5-22 (2017).
51. Sun, B. B., et al. Genomic atlas of the human plasma proteome. Nature 558, 73-79 (2018).
52. Sekula, P., Del Greco, M. F., Pattaro, C. & Kottgen, A. Mendelian Randomization as an Approach to Assess Causality Using Observational Data. J Am Soc Nephrol 27, 3253-3265 (2016).
53. Tragante, V. & Asselbergs, F. W. Mendelian randomization: A powerful method to determine causality of biomarkers in diseases. Int J Cardiol 268, 227-228 (2018).
54. Do, R., et al. Common variants associated with plasma triglycerides and risk for coronary artery disease. Nat Genet 45, 1345-1352 (2013).
55. Folkersen, L., et al. Genomic and drug target evaluation of 90 cardiovascular proteins in 30,931 individuals. Nat Metab 2, 1135-1148 (2020).
56. Getz, G. S. & Reardon, C. A. Animal models of atherosclerosis. Arterioscler Thromb Vasc Biol 32, 1104-1115 (2012).
57. Oppi, S., Luscher, T. F. & Stein, S. Mouse Models for Atherosclerosis Research-Which Is My Line? Front Cardiovasc Med 6, 46 (2019).
58. Kim, E. Y. & Dryer, S. E. RAGE and alphaVbeta3-integrin are essential for suPAR signaling in podocytes. Biochim Biophys Acta Mol Basis Dis 1867, 166186 (2021).
59. Jenkins, W. S., et al. In vivo alpha-V beta-3 integrin expression in human aortic atherosclerosis. Heart 105, 1868-1875 (2019).
60. Winter, P. M., et al. Molecular imaging of angiogenesis in early-stage atherosclerosis with alpha(v)beta3-integrin-targeted nanoparticles. Circulation 108, 2270-2274 (2003).
61. Antonov, A. S., et al. alphaVbeta3 integrin regulates macrophage inflammatory responses via PI3 kinase/Akt-dependent NF-kappaB activation. J Cell Physiol 226, 469-476 (2011).
62. Antonov, A. S., Kolodgie, F. D., Munn, D. H. & Gerrity, R. G. Regulation of macrophage foam cell formation by alphaVbeta3 integrin: potential role in human atherosclerosis. Am J Pathol 165, 247-258 (2004).
63. Winnicki, W., et al. Diagnostic and Prognostic Value of Soluble Urokinase-type Plasminogen Activator Receptor (suPAR) in Focal Segmental Glomerulosclerosis and Impact of Detection Method. Sci Rep 9, 13783 (2019).
64. Hayek, S., et al. Assay-related Differences in SuPAR Levels: Implications for Measurement and Data Interpretation. medRxiv, 2021.2006.2023.21259148 (2021).
65. Raffield, L. M., et al. Comparison of Proteomic Assessment Methods in Multiple Cohort Studies. Proteomics 20, e1900278 (2020).
66. Joshi, A. & Mayr, M. In Aptamers They Trust: The Caveats of the SOMAscan Biomarker Discovery Platform from SomaLogic. Circulation 138, 2482-2485 (2018).
67. Abraham, A. G., et al. Variability in CKD Biomarker Studies: suPAR and Kidney Disease Progression in the Chronic Kidney Disease in Children (CKiD) Study. Kidney Medicine (2021).
68. Olson, N. C., et al. Soluble Urokinase Plasminogen Activator Receptor: Genetic Variation and Cardiovascular Disease Risk in Blacks. Circulation: Genomic and Precision Medicine, Circgen121003421 (2021).
69. von Scheidt, M., et al. Applications and Limitations of Mouse Models for Understanding Human Atherosclerosis. Cell Metab 25, 248-261 (2017).
70. Ridker, P. M., et al. Antiinflammatory Therapy with Canakinumab for Atherosclerotic Disease. N Engl J Med 377, 1119-1131 (2017).
71. Ridker, P. M., et al. IL-6 inhibition with ziltivekimab in patients at high atherosclerotic risk (RESCUE): a double-blind, randomised, placebo-controlled, phase 2 trial. Lancet 397, 2060-2069 (2021).
72. Staeck, O., et al. Recurrent Primary Focal Segmental Glomerulosclerosis Managed With Intensified Plasma Exchange and Concomitant Monitoring of Soluble Urokinase-Type Plasminogen Activator Receptor-Mediated Podocyte beta3-integrin Activation. Transplantation 99, 2593-2597 (2015).
73. Alachkar, N., et al. Podocyte effacement closely links to suPAR levels at time of posttransplantation focal segmental glomerulosclerosis occurrence and improves with therapy. Transplantation 96, 649-656 (2013).
74. Alachkar, N., et al. Monitoring suPAR levels in post-kidney transplant focal segmental glomerulosclerosis treated with therapeutic plasma exchange and rituximab. BMC Nephrol 19, 361 (2018).
75. Bild, D. E., et al. Multi-Ethnic Study of Atherosclerosis: Objectives and Design. American Journal of Epidemiology 156, 871-881 (2002).
76. Carr, J. J., et al. Calcified Coronary Artery Plaque Measurement with Cardiac CT in Population-based Studies: Standardized Protocol of Multi-Ethnic Study of Atherosclerosis (MESA) and Coronary Artery Risk Development in Young Adults (CARDIA) Study. Radiology 234, 35-43 (2005).
77. Breen, J. F., et al. Coronary artery calcification detected with ultrafast CT as an indication of coronary artery disease. Radiology 185, 435-439 (1992).
78. Carr, J. J., et al. Coronary artery calcium quantification with retrospectively gated helical CT: Protocols and techniques. The International Journal of Cardiovascular Imaging 17, 213-220 (2001).
79. Nelson, J. C., et al. Measuring Coronary Calcium on CT Images Adjusted for Attenuation Differences. Radiology 235, 403-414 (2005).
80. Riisbro, R., Christensen, I. J., Hogdall, C., Brunner, N. & Hogdall, E. Soluble urokinase plasminogen activator receptor measurements: influence of sample handling. Int J Biol Markers 16, 233-239 (2001).
81. Wei, C., et al. Circulating suPAR in two cohorts of primary FSGS. J Am Soc Nephrol 23, 2051-2059 (2012).
82. Levey, A. S., et al. A new equation to estimate glomerular filtration rate. Ann Intern Med 150, 604-612 (2009).
83. Haas, M. E., et al. Genetic Association of Albuminuria with Cardiometabolic Disease and Blood Pressure. Am J Hum Genet 103, 461-473 (2018).
84. Mills, J. L., et al. Do high blood folate concentrations exacerbate metabolic abnormalities in people with low vitamin B-12 status? Am J Clin Nutr 94, 495-500 (2011).
85. Desch, K. C., et al. Linkage analysis identifies a locus for plasma von Willebrand factor undetected by genome-wide association. Proc Natl Acad Sci USA 110, 588-593 (2013).
86. Bild, D. E., et al. Multi-Ethnic Study of Atherosclerosis: objectives and design. Am J Epidemiol 156, 871-881 (2002).
87. Berglund, G., Elmstahl, S., Janzon, L. & Larsson, S. A. The Malmo Diet and Cancer Study. Design and feasibility. J Intern Med 233, 45-51 (1993).
88. Hansen, T. F., et al. DBDS Genomic Cohort, a prospective and comprehensive resource for integrative and temporal analysis of genetic, environmental and lifestyle factors affecting health of blood donors. BMJ Open 9, e028401 (2019).
89. McCarthy, S., et al. A reference panel of 64,976 haplotypes for genotype imputation. Nat Genet 48, 1279-1283 (2016).
90. Das, S., et al. Next-generation genotype imputation service and methods. Nat Genet 48, 1284-1287 (2016).
91. Zhao, H., et al. CrossMap: a versatile tool for coordinate conversion between genome assemblies. Bioinformatics 30, 1006-1007 (2014).
92. Chang, C. C., et al. Second-generation PLINK: rising to the challenge of larger and richer datasets. Gigascience 4, 7 (2015).
93. Zhan, X., Hu, Y., Li, B., Abecasis, G. R. & Liu, D. J. RVTESTS: an efficient and comprehensive tool for rare variant association analysis using sequence data. Bioinformatics 32, 1423-1426 (2016).
94. Willer, C. J., Li, Y. & Abecasis, G. R. METAL: fast and efficient meta-analysis of genomewide association scans. Bioinformatics 26, 2190-2191 (2010).
95. McLaren, W., et al. The Ensembl Variant Effect Predictor. Genome Biol 17, 122 (2016).
96. Sudlow, C., et al. UK biobank: an open access resource for identifying the causes of a wide range of complex diseases of middle and old age. PLoS Med 12, e1001779 (2015).
97. Bycroft, C., et al. The UK Biobank resource with deep phenotyping and genomic data. Nature 562, 203-209 (2018).
98. Pirruccello, J. P., et al. Analysis of cardiac magnetic resonance imaging in 36,000 individuals yields genetic insights into dilated cardiomyopathy. Nat Commun 11, 2254 (2020).
99. Wang, Q., et al. Rare variant contribution to human disease in 281,104 UK Biobank exomes. Nature 597, 527-532 (2021).
100. Bjorklund, M. M., et al. Induction of atherosclerosis in mice and hamsters without germline genetic engineering. Circ Res 114, 1684-1689 (2014).
101. Daugherty, A., et al. Recommendation on Design, Execution, and Reporting of Animal Atherosclerosis Studies: A Scientific Statement From the American Heart Association. Arterioscler Thromb Vasc Biol 37, e131-e157 (2017).
102. Paigen, B., Morrow, A., Holmes, P. A., Mitchell, D. & Williams, R. A. Quantitative assessment of atherosclerotic lesions in mice. Atherosclerosis 68, 231-240 (1987).

1. A method of treating or preventing atherosclerosis in a subject, comprising treating the subject to reduce soluble urokinase plasminogen activator receptor (suPAR) protein levels and/or inhibit the activity or downstream effectors of suPAR.

2. The method of claim 1, wherein the subject exhibits elevated levels of suPAR.

3. The method of claim 1, wherein the subject exhibits elevated levels of suPAR in the blood.

4. The method of claim 1, wherein the subject suffers from atherosclerosis.

5. The method of claim 1, wherein the subject is at elevated risk of atherosclerosis based on one or more risk factors.

6. The method of claim 1, wherein treating the subject to reduce suPAR protein levels and/or inhibit the activity of suPAR comprises administering an anti-suPAR therapy to the subject.

7. The method of claim 6, wherein the anti-suPAR therapy inhibits the expression of suPAR.

8. The method of claim 7, wherein the anti-suPAR therapy is a nucleic acid inhibitor of suPAR expression.

9. The method of claim 8, wherein the nucleic acid inhibitor of suPAR expression is an antisense oligonucleotide (ASO), an siRNA, an shRNA, or an element of a Cas/CRISPR system.

10. The method of claim 6, wherein the anti-suPAR therapy inhibits the activity of suPAR.

11. The method of claim 10, wherein the anti-suPAR therapy is an antibody or antibody fragment that binds to suPAR or a ligand or receptor of suPAR and thereby inhibits the activity of suPAR.

12. The method of claim 10, wherein the anti-suPAR therapy is a peptide or small molecule that binds to suPAR or a downstream effector of suPAR activity and thereby inhibits the function of suPAR.

13. The method of claim 12, wherein the small molecule is azeliragon or other RAGE antagonists.

14. A method of assessing and treating/preventing atherosclerosis in a subject, comprising:
(a) determining the level of soluble urokinase plasminogen activator receptor (suPAR) protein in a sample obtained from a subject; and
(b) treating the subject to reduce suPAR levels and/or inhibit suPAR activity if the suPAR level in the sample is elevated.

15. The method of claim 14, wherein the sample is a blood sample or a processed blood sample.

16. The method of claim 15, comprising a step of comparing the level of suPAR to a threshold level to determine if the level of suPAR is elevated.

17. The method of claim 16, wherein the threshold level is based on an algorithm that incorporates at least the age, renal function, and gender of the subject.

18. The method of claim 14, wherein treating the subject to reduce excess suPAR levels and/or inhibit suPAR activity comprises administering an anti-suPAR therapy to the subject.

19. The method of claim 18, wherein the anti-suPAR therapy is (1) administration of an anti-suPAR small molecule, peptide, or antibody treatment which specifically binds to suPAR, (2) administration of a small molecule, peptide, or antibody treatment which specifically binds to a ligand or receptor of suPAR, or (3) plasmapheresis to remove suPAR from the subject.

20. The method of claim 20, wherein the anti-suPAR therapy inhibits the expression of suPAR.

21. The method of claim 20, wherein the anti-suPAR therapy is a nucleic acid inhibitor of suPAR expression.

22. The method of claim 21, wherein the nucleic acid inhibitor of suPAR expression is an antisense oligonucleotide (ASO), an siRNA, an shRNA, or an element of a Cas/CRISPR system.

23. The method of claim 20, wherein the anti-suPAR therapy inhibits the activity of suPAR.

24. The method of claim 23, wherein the anti-suPAR therapy is an antibody or antibody fragment that binds to suPAR or a ligand or receptor of suPAR and thereby inhibits the activity of suPAR.

25. The method of claim 23, wherein the anti-suPAR therapy is a peptide or small molecule that binds to suPAR and thereby inhibits the activity of suPAR.

26. The method of claim 25, wherein the small molecule is azeliragon or other RAGE antagonists


SEQUENCE LISTING (6 sequences)

SEQ ID NO: 1 - protein, 5 aa, Homo sapiens
SRSRY

SEQ ID NO: 2 - protein, 335 aa, Homo sapiens
MGHPPLLPLLLLLHTCVPASWGLRCMQCKTNGDCRVEECALGQDLCRTTIVRLWEEGEELELVEKSCTHSEKTNRTLSYRTGLKITSLTEVVCGLDLCNQGNSGRAVTYSRSRYLECISCGSSDMSCERGRHQSLQCRSPEEQCLDVVTHWIQEGEEGRPKDDRHLRGCGYLPGCPGSNGFHNNDTFHFLKCCNTTKCNEGPILELENLPQNGRQCYSCKGNSTHGCSSEETFLIDCRGPMNQCLVATGTHEPKNQSYMVRGCATASMCQHAHLGDAFSMNHIDVSCCTKSGCNHPDLDVQYRSGAAPQPGPAHLSLTITLLMTARLWGGTLLWT

SEQ ID NO: 3 - DNA, 43 bp, Homo sapiens
  source 1..43: mol_type = genomic DNA
ccatgggtgctgttccccttgcagctgtaacactggcggccat

SEQ ID NO: 4 - DNA, 38 bp, Homo sapiens
  source 1..38: mol_type = genomic DNA
ggcagtcattagcagggtgatggtgaggctgagatggg

SEQ ID NO: 5 - DNA, 43 bp, Homo sapiens
  source 1..43: mol_type = genomic DNA
ccatgggtgctgttccccctgcagctgtaacactggcggccat

SEQ ID NO: 6 - DNA, 37 bp, Homo sapiens
  source 1..37: mol_type = genomic DNA
gcagtcattagcagggtgatggtggggctgagatggg